SEQUENCE LISTING (22 sequences)

SEQ ID NO: 1 - protein, 483 aa, Bacillus
HHNGTNGTLMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVKAVEVNPNNRNQEVSGEYTIEAWTKFDFPGRANTHSNFKWRWYHFDGVDWDQSRKLNNRIYKFRTKAWDWEVDTEFGNYDYLLYADIDMDHPEVVNELRNWGVWYTNTLGLDGFRIDAVKHIKYSFTRDWINHVRSAIGKNMFAVAEFWKNDLGAIENYLNKTNWNHSVFDVPLHFNLYYASKSGGNYDMRQIFNGTVVQKHPTHAVTFVDNHDSQPEESLESFVREWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNKAGTVTINADGWGNFSVNGGSVSIWVNK

SEQ ID NO: 2 - protein, 485 aa, Bacillus
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 3 - protein, 485 aa, Bacillus sp 707
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 4 - protein, 399 aa, Synthetic Construct
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYG

SEQ ID NO: 5 - protein, 86 aa, Synthetic Construct
KQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 6 - protein, 483 aa, Bacillus amyloliquefaciens
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 7 - protein, 397 aa, Synthetic Construct
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYG

SEQ ID NO: 8 - protein, 85 aa, Synthetic Construct
PQHDYIDHPDVIGWTREGDSSAAKSGLAALITDGPGGSKRMYAGLKNAGTWYDITGNRSDTVKIGSDGWGEFHVNDGSVSIYVQK

SEQ ID NO: 9 - protein, 483 aa, Synthetic Construct
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 10 - protein, 514 aa, Bacillus
MKQQKRLYARLLTLLFALIFLLPHSAAAAHHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQDKAGQVWHDITGNKPGTVTINADGWANFSVNGGSVSIWVKR

SEQ ID NO: 11 - protein, 485 aa, Bacillus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQDKAGQVWHDITGNKPGTVTINADGWANFSVNGGSVSIWVKR

SEQ ID NO: 12 - protein, 483 aa, Bacillus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQDKAGQVWHDITGNKPGTVTINADGWANFSVNGGSVSIWVKR

SEQ ID NO: 13 - protein, 513 aa, Alicyclobacillus-FL
MKIRNGWKKTLTLLFALIFLLPHSAAATFAGDNGTMMQYFEWYLPNDGTLWTKMGSDASHLKSIGITGVWFPPAYKGQSQSDVGYGVYDMYDLGEFNQKGTVRTKYGTKAQLQSAITSLHNNGIQAYGDVVLNHRMGADATETISAVEVNPSNRNQVTSGAYNISAWTDFEFPGRGNTYSSFKWHSYYFDGVDWDQSRQLSGKIYQIQGTGKAWDWEVDSENGNYDYLMGADIDYDHPDVQTEVKNWGKWFVNTLNLDGVRLDAVKHIKFDYMSSWLSSVKSTTGKSNLFAVGEYWNTSLGALENYENKTNWSMSLFDVPLHMNFQAAANGGGYYDMRNLLNNTMMKNHPIQAVTFVDNHDTEPGQALQSWVSDWFKPLAYATILTRQEGYPCVFYGDYYGIPSQSVSAKSTWLDKQLSARKSYAYGTQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 14 - protein, 486 aa, Alicyclobacillus sp
TFAGDNGTMMQYFEWYLPNDGTLWTKMGSDASHLKSIGITGVWFPPAYKGQSQSDVGYGVYDMYDLGEFNQKGTVRTKYGTKAQLQSAITSLHNNGIQAYGDVVLNHRMGADATETISAVEVNPSNRNQVTSGAYNISAWTDFEFPGRGNTYSSFKWHSYYFDGVDWDQSRQLSGKIYQIQGTGKAWDWEVDSENGNYDYLMGADIDYDHPDVQTEVKNWGKWFVNTLNLDGVRLDAVKHIKFDYMSSWLSSVKSTTGKSNLFAVGEYWNTSLGALENYENKTNWSMSLFDVPLHMNFQAAANGGGYYDMRNLLNNTMMKNHPIQAVTFVDNHDTEPGQALQSWVSDWFKPLAYATILTRQEGYPCVFYGDYYGIPSQSVSAKSTWLDKQLSARKSYAYGTQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 15 - protein, 485 aa, Bacillus
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGRQHDYFDHWDVIGWTREGNASHPNSGLATIMSDGPGGSKWMYVGRQKAGEVWHDMTGNRSGTVTINQDGWGHFFVNGGSVSVWVKR

SEQ ID NO: 16 - protein, 483 aa, Bacillus
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGRQHDYFDHWDVIGWTREGNASHPNSGLATIMSDGPGGSKWMYVGRQKAGEVWHDMTGNRSGTVTINQDGWGHFFVNGGSVSVWVKR

SEQ ID NO: 17 - protein, 399 aa, Bacillus
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYG

SEQ ID NO: 18 - protein, 86 aa, Alicyclobacillus
TQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 19 - protein, 485 aa, Synthetic Construct
HHDGTNGTIMQYFEWNVPNDGQHWNRLHNNAQNLKNAGITAIWIPPAWKGTSQNDVGYGAYDLYDLGEFNQKGTVRTKYGTKAELERAIRSLKANGIQVYGDVVMNHKGGADFTERVQAVEVNPQNRNQEVSGTYQIEAWTGFNFPGRGNQHSSFKWRWYHFDGTDWDQSRQLANRIYKFRGDGKAWDWEVDTENGNYDYLMYADVDMDHPEVINELNRWGVWYANTLNLDGFRLDAVKHIKFSFMRDWLGHVRGQTGKNLFAVAEYWKNDLGALENYLSKTNWTMSAFDVPLHYNLYQASNSSGNYDMRNLLNGTLVQRHPSHAVTFVDNHDTQPGEALESFVQGWFKPLAYATILTREQGYPQVFYGDYYGIPSDGVPSYRQQIDPLLKARQQYAYGTQHDYLDNQDVIGWTREGDSAHAGSGLATVMSDGPGGSKTMYVGTAHAGQVFKDITGNRTDTVTINSAGNGTFPCNGGSVSIWVKQ

SEQ ID NO: 20 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 21 - protein, 275 aa, Bacillus amyloliqufacens [sequence omitted]

SEQ ID NO: 22 - protein, 311 aa, Bacillus [sequence omitted]

(19) United States
(12) Patent Application Publication
(10) Pub. No.: US 2022/0017844 A1
(43) Pub. Date: Jan. 20, 2022

(54) Low pH Powder Detergent Composition

(71) Applicant: Novozymes A/S, Bagsvaerd (DK)

(72) Inventors: Astrid Benie, Vaerloese (DK); Pascal Schauwers, Bousval (BE); Elena Genesca Pont, Copenhagen (DK); Maria Norman Hockauf, Stenloese (DK)

(73) Assignee: Novozymes A/S, Bagsvaerd (DK)

(21) Appl. No.: 17/299,261

(22) PCT Filed: Dec. 2, 2019
(86) PCT No.: PCT/EP2019/083304

§ 371 Date: Jun. 2, 2021

(30) Foreign Application Priority Data
Dec. 3, 2018 (EP) 18209710.5
Dec. 3, 2018 (EP) 18209711.3
Dec. 3, 2018 (EP) 18209714.7
Aug. 19, 2019 (EP) 19192257.4

(51) Int. Cl. C11D 17/06; C11D 3/00; C11D 3/386; C11D 11/00

(52) U.S. Cl. CPC C11D 17/06; C11D 3/0047; C11D 11/0035; C11D 11/0017; C11D 3/38609

(57) ABSTRACT

The invention relates to low pH powder detergent composition comprising at least one polypeptide having alpha-amylase activity and at least 60%, such as 65%, such as 70%, such as 75%, such as 80%, such as 85%, such as 90%, such as 95%, such as 97% sequence identity to SEQ ID NOs: 1-19 and the composition has a pH value below 9.5.

### REFERENCE TO SEQUENCE LISTING

This application contains a Sequence Listing in computer readable form. The computer readable form is incorporated herein by reference.

### FIELD OF THE INVENTION

The present invention relates to powder detergent compositions, in particular to low pH powder detergent compositions comprising at least one polypeptide having an alpha-amylase.

### BACKGROUND OF THE INVENTION

Alpha-amylases have for many years been used in laundry where is it well-known that alpha-amylases have a beneficial effect in removal of starch containing, or starch-based, stains.

WO95/26397 discloses alkaline Bacillus amylases having good wash performance measured at temperatures in the range of 30-60° C.

WO00/60060 and WO00/60058 discloses further bacterial alpha-amylases having good wash performance.

WO2016/180748 discloses alpha-amylase variants have improves wash performance at low temperatures. It was demonstrated that the variants had good performance in liquid detergent compositions.

Detergents compositions may be provided in different formulations such as liquid formulations and powder formulations.

Since the ingredients in powder formulations are in a solid form with low water content, enzymes in powder formulations are also in solid form and are in this form, at least to some degree, protected against proteolytic degradation. Powder detergent formulations are typically highly alkaline and pH values above 9.0, and even a pH value above 10.0 are not unusual. Further, powder detergents may contain bleach.

Liquid detergent formulation typically contains water and the enzymes therein are typically in a soluble form which means that enzymes in liquid detergents are more susceptible to degradation such as proteolytic degradation, compared with enzymes in powder formulations.

As consequence of the significant differences between powder detergents and liquid detergent formulations, different enzymes are typically used in these two formulations forms, and it cannot be concluded that because an enzyme performs well in e.g. powder detergent formulations it also performs well in liquid detergent formulations and vice versa.

### SUMMARY OF THE INVENTION

The present invention relates to low pH powder detergent composition comprising at least one polypeptide having alpha-amylase activity, where the presence of an alpha-amylase in the composition has surprisingly been found to resulting in an improved cleaning performance.

The invention provides a powder detergent composition comprising at least one polypeptide having alpha-amylase activity and having at least 60%, such as 65%, such as 70%, such as 75%, such as 80%, such as 85%, such as 90%, such as 95%, such as 97% sequence identity to SEQ ID NOs: 1-19 and wherein the composition has a pH value below 9.5, e.g. in the range of 7.0-9.5; e.g. in the range of 7.5 to 9.0; e.g. in the range of 8.0 to 9.0.

The present invention also relates to use of a composition described herein in a cleaning process, e.g. for laundry or dishwashing, and to a method of cleaning using the low pH detergent composition.

### Definitions

Dish washing composition: The term “dish washing composition” refers to compositions intended for cleaning dishes, table ware, pots, pans, cutlery and all forms of compositions for cleaning hard surfaces areas in kitchens. The present invention is not restricted to any particular type of dish wash composition or any particular detergent.
Enzyme Detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and/or cleaning, prevention or reduction of redeposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of redeposition of soils, are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibres from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyse the formation of bleaching components such as hydrogen peroxide or other peroxides.
Improved wash performance: The term “improved wash performance” is defined herein as an increased wash performance relative to the wash performance of a detergent composition e.g. by increased stain removal. The term “improved wash performance” includes wash performance in laundry.
Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, cutlery such as spoons, knives, forks, serving utensils, ceramics, plastics, metals, china, glass and acrylics.
Powder detergent composition: The term “powder detergent composition” is defined herein as a detergent composition wherein all or most of the ingredients are in solid dry form. Powder typically consists of a mixture comprising one or more powders and or granulates. The term powder detergent composition includes unit dosage forms such as tabs, tablets, that have been made by combining, pressing or agglomerating one or more powders into a larger structure and which appears in a dry form. Thus, the water content in a powder detergent composition should be sufficiently low to prevent stickiness or unintended agglomeration of the composition into larger structures. The present description and claims will often refer to a “powder” composition for the sake of simplicity. Unless otherwise indicated or apparent from the context, the term “powder” as used herein should be understood to also include solid forms such as granulates and tabs as described above.
pH of a powder detergent composition: The pH of a powder detergent composition is intended to mean the pH at 20° C. of an aqueous ready-to-use solution of the powder detergent composition in water. In order to measure the pH of a powder detergent composition the first step is to prepare a solution of 5 g of the powder detergent composition per liter water, followed by measuring pH in the solution using well known techniques and/or equipment for pH measurements.

In case that the powder detergent composition is in form of a unit dosage form, one-unit dose is dissolved in 15 liters of water and the pH at 20° C. of this solution is measured and this pH value is considered to be the pH of the powder detergent composition in unit dose form.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used it is intended to include the broader term textiles as well.
Variant: The term “variant” means a polypeptide having alpha-amylase activity comprising an alteration, i.e., a substitution, insertion, and/or deletion, at one or more positions. A substitution means replacement of the amino acid occupying a position with a different amino acid; a deletion means removal of the amino acid occupying a position; and an insertion means adding an amino acid adjacent to and immediately following the amino acid occupying a position.
Wash performance: The term “wash performance” is used as an enzyme's ability to remove stains present on the object to be cleaned during e.g. wash or hard surface cleaning. The improvement in the wash performance may be quantified by calculating the so-called intensity value (Int), herein. The term “wash performance” and “dish wash performance” are used interchangeably.

### Conventions for Designation of Variants

For purposes of the present invention, the polypeptide disclosed in SEQ ID NO: 1 is used to determine the corresponding amino acid residue in another alpha-amylase. The amino acid sequence of another alpha-amylase is aligned with the polypeptide disclosed in SEQ ID NO: 1, and based on the alignment, the amino acid position number corresponding to any amino acid residue in the polypeptide disclosed in SEQ ID NO: 1 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another alpha-amylase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

For proteins of known structure, several tools and resources are available for retrieving and generating structural alignments. For example, the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants of the present invention, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions: For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Multiple modifications: Variants comprising multiple modifications are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different modifications: Where different modifications can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

The present invention relates in one aspect to a powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NOs: 1-19 and wherein the composition has a pH value below 9.5.

In one embodiment, the present invention relates to a composition having a pH of not more than about 9, such as in the range of 7.0-9.5; such as in the range of 7.5 to 9.0; such as in the range of 8.0 to 9.0.

It will be apparent from the present description that the term “low pH” as used herein refers to a lower pH relative to conventional powder detergents such as those used for laundry or dishwash, which as mentioned above typically have a pH in use of above 9 and often above 10.

As indicated above, the term “powder” as used herein is understood to refer to a composition in solid dry form. The “powders” of the invention typically consist of a mixture comprising one or more powders and/or granulates, but also include e.g. unit dosage forms such as tabs.

In one embodiment, the composition of the invention has a pH of below about 9.5, such as not more than about 9.4, such as not more than about 9.3, such as not more than about 9.2, such as not more than about 9.1, such as not more than about 9.0, such as not more than about 8.9, such as not more than about 8.8, such as not more than about 8.7, such as not more than about 8.6, such as not more than about 8.5, such as not more than about 8.4, such as not more than about 8.3, such as not more than about 8.2, such as not more than about 8.1, or not more than about 8.0. On the other hand, the composition will generally have a pH of at least about 7, such as at least about 7.1, at least about 7.2, at least about 7.3, at least about 7.4, at least about 7.5, at least about 7.6, at least about 7.7, at least about 7.8, or at least about 7.9. In all cases, pH is determined in a 5 g/l solution as described above.

In some embodiments, the pH may e.g. be in the range of from about 7.0 to not more than about 9.0, for example from about 7.2 to about 8.9, such as from about 7.4 to about 8.8, such as from about 7.6 to about 8.7, such as from about 7.8 to about 8.6.

In one embodiment, the pH may be in the range of from about 7.0 to about 8.2, such as from about 7.2 to about 8.0, determined in a 5 g/l solution as described above.

In another embodiment, the pH may be in the range of from about 7.8 to about 8.8, such as from about 8.0 to about 8.6, determined in a 5 g/l solution as described above.

It should be noted that although, as described above, pH is generally determined in a 5 g/l solution, it is contemplated that for unit dosage forms, e.g. tabs, pH may be determined by dissolving one unit, e.g. one tab, in 15 l of deionized water at 20° C. and measuring the pH of this solution.

The powder detergent composition of the invention has a low pH value. As will be appreciated by the skilled person, pH values are in general measured in an aqueous solution and not in a dry powder, so it should be understood that the pH value of a dry composition such as a powder detergent composition in this disclosure is intended to mean the pH value of an aqueous solution of the composition in question. Thus, according to the invention the pH of a powder detergent composition is determined by preparing a 5 g per liter solution of the powder detergent composition in water and after the detergent composition has been completely dissolved in water measuring the pH value using techniques known in the art for pH measurement.

It should be noted that although, as described above, pH is generally determined in a 5 g/l solution, it is contemplated that for unit dosage forms, e.g. tabs, pH may be determined by dissolving one unit, e.g. one tab, in 151 of deionized water at 20° C. and measuring the pH of this solution.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NOs: 1-19.

In one aspect, the invention relates to a low pH powder detergent composition comprising at least two polypeptide having alpha-amylase activity and having at least 60%, at least 65%, at least 70%, at least 75%, at least 80%, at least 85%, at least 90% or at least 95% but less than 100% sequence identity to SEQ ID NOs: 1-19.

In one aspect, the invention relates to a low pH powder detergent composition comprising at least three polypeptide having alpha-amylase activity and having at least 60%, at least 65%, at least 70%, at least 75%, at least 80%, at least 85%, at least 90% or at least 95% but less than 100% sequence identity to SEQ ID NOs: 1-19.

In one aspect, the invention relates to a low pH powder detergent composition comprising at least four polypeptide having alpha-amylase activity and having at least 60%, at least 65%, at least 70%, at least 75%, at least 80%, at least 85%, at least 90% or at least 95% but less than 100% sequence identity to SEQ ID NOs: 1-19.

In one aspect, the invention relates to a low pH powder detergent composition comprising at least five polypeptide having alpha-amylase activity and having at least 60%, at least 65%, at least 70%, at least 75%, at least 80%, at least 85%, at least 90% or at least 95% but less than 100% sequence identity to SEQ ID NOs: 1-19.

In one aspect, the invention relates to a low pH powder detergent composition comprising at least one or more polypeptide having alpha-amylase activity and having at least 60%, at least 65%, at least 70%, at least 75%, at least 80%, at least 85%, at least 90% or at least 95% but less than 100% sequence identity to SEQ ID NOs: 1-19.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 1.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 2.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 3.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 4.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 5.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 6.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 7.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 8.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 9.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 10.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 11.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 12.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 13.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 14.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 15.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 16.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 17.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 18.

In one aspect, the invention relates to a low pH powder detergent composition comprising at least one polypeptide having alpha-amylase activity and having at least 60%, at least 65%, at least 70%, at least 75%, at least 80%, at least 85%, at least 90% or at least 95% but less than 100% sequence identity to SEQ ID NO: 19.

In one aspect, the invention relates to a low pH powder detergent composition comprising a variant of a polypeptide having alpha-amylase activity and having at least 60%, at least 65%, at least 70%, at least 75%, at least 80%, at least 85%, at least 90% or at least 95% but less than 100% sequence identity to SEQ ID NOs: 1-19.

In one embodiment, the invention relates to a variant of a polypeptide having alpha-amylase activity comprising an alteration at two or more (several) positions corresponding to positions G304, W140, W189, D134, E260, F262, W284, W347, W439, W469, G476, and G477 of the polypeptide of SEQ ID NO: 11, wherein each alteration is independently a substitution, deletion or insertion, and wherein the variant has at least 60%, at least 65%, at least 70%, at least 75%, at least 80%, or at least 87%, but less than 100% sequence identity with the polypeptide of SEQ ID NO: 11, and wherein the variant has alpha-amylase activity.

In one embodiment, the polypeptide having alpha-amylase activity is a variant of SEQ ID NO: 11 comprising substitutions selected from the group consisting of: W140Y+N195F+V206Y+Y243F+E260G+G477E, W140Y+N195F+V206Y+Y243F+E260T+W284D, W140Y+N195F+V206Y+Y243F+W284D, G109A+W140Y+N195F+V206Y+Y243F+E260G, W140Y+N195F+V206Y+Y243F+E260G, N195F+V206Y+Y243F+E260K+W284D, D134E+G476E, W140Y+N195F+V206Y+Y243F+E260G+G476E, W140Y+W189G+N195F+V206Y+Y243F+E260G, W140Y+N195F+V206Y+Y243F+E260G+S303G, W140Y+W189T+N195F+V206Y+Y243F+E260G, W140Y+N195F+V206Y+Y243F+E260G+W284D, Y100I+W140Y+N195F+V206Y+Y243F+E260G, W140Y+N195F+V206Y+Y243F+E260G+G337N, W140Y+N195F+V206Y+Y243F+E260G+W439R G109A+W140Y+E194D+N195F+V206Y+Y243F+E260G G109A+W140Y+N195F+V206Y+Y243F+E260G+G476E T51I+Y100I+G109A+W140Y+N195F+V206Y+Y243F+E260G T51I+G109A+W140Y+N195F+V206Y+Y243F+E260G+W439R T51I+S52Q+N54K+G109A+W140Y+N195F+V206Y+Y243F+E260G+G476E W140Y+N195F+V206Y+Y243F+E260G+G304R+G476K W140Y+N195F+V206Y+Y243F+E260G+W284R+G477K W140Y+N195F+V206Y+Y243F+E260G+W284F+G477R, and N195F+V206Y+Y243F+E260G+W284D.

Preferably, the variant further comprises deletions at positions corresponding to positions G182*+D183* or D183*+G184* of SEQ ID NO:11.

Preferred examples of variants to be used in the compositions of the invention comprises variants which comprises or consists of alterations in the positions, selected from the group selected among: D183*+G184*+W140Y+N195F+V206Y+Y243F+E260G+G477E, D183*+G184*+W140Y+N195F+V206Y+Y243F+E260T+W284D, D183*+G184*+W140Y+N195F+V206Y+Y243F+W284D, D183*+G184*+G109A+W140Y+N195F+V206Y+Y243F+E260G, D183*+G184*+W140Y+N195F+V206Y+Y243F+E260G, D183*+G184*+N195F+V206Y+Y243F+E260K+W284D, D183*+G184*+D134E+G476E, D183*+G184*+W140Y+N195F+V206Y+Y243F+E260G+G476E, D183*+G184*+W140Y+W189G+N195F+V206Y+Y243F+E260G, D183*+G184*+W140Y+N195F+V206Y+Y243F+E260G+S303G, D183*+G184*+W140Y+W189T+N195F+V206Y+Y243F+E260G, D183*+G184*+W140Y+N195F+V206Y+Y243F+E260G+W284D, D183*+G184*+Y100I+W140Y+N195F+V206Y+Y243F+E260G, D183*+G184*+W140Y+N195F+V206Y+Y243F+E260G+G337N, D183*+G184*+W140Y+N195F+V206Y+Y243F+E260G+W439R D183*+G184*+G109A+W140Y+E194D+N195F+V206Y+Y243F+E260G D183*+G184*+G109A+W140Y+N195F+V206Y+Y243F+E260G+G476E D183*+G184*+T51I+Y100I+G109A+W140Y+N195F+V206Y+Y243F+E260G D183*+G184*+T51I+G109A+W140Y+N195F+V206Y+Y243F+E260G+W439R D183*+G184*+T51I+S52Q+N54K+G109A+W140Y+N195F+V206Y+Y243F+E260G+G476E D183*+G184*+W140Y+N195F+V206Y+Y243F+E260G+G304R+G476K D183*+G184*+W140Y+N195F+V206Y+Y243F+E260G+W284R+G477K D183*+G184*+W140Y+N195F+V206Y+Y243F+E260G+W284F+G477R, and D183*+G184*+N195F+V206Y+Y243F+E260G+W284D, H1*+G109A+W140Y+D183*+G184*+N195F+1206Y+Y243F+E260G+N280S+G304R+E391A+G476K, H1*+G109A+W140Y+D183*+G184*+N195F+1206Y+Y243F+E260G+W284H+G304R+E391A+G476K, H1*+G109A+W140Y+D183*+G184*+N195F+1206Y+Y243F+E260G+N280S+G304R+K320A+M323N+E391A+G476K, H1*+G7A+G109A+W140Y+D183*+G184*+N195F+1206Y+Y243F+E260G+N280S+G304R+E391A+G476K, and H1*+G7A+G109A+W140Y+D183*+G184*+N195F+1206Y+Y243F+E260G+N280S+W284H+G304R+M323N+E391A+G476K.

In one embodiment, the polypeptide having alpha-amylase activity in the powder detergent composition of the invention includes the variants of SEQ ID NO: 11 comprising or consisting of the alterations: D183*+G184*+W140Y+N195F+V206Y+Y243F+E260G+G304R+G476K, and H1*+G7A+G109A+W140Y+D183*+G184*+N195F+1206Y+Y243F+E260G+N280S+G304R+E391A+G476K.

In one embodiment, the invention relates to a variant of a polypeptide having alpha-amylase activity comprising a mutation at one or more (e.g., several) positions within the amino acid sequence of the polypeptide of SEQ ID NO: 1, wherein said variant amino acid sequence comprises a mutation at amino acid position 202 and/or 186 wherein numbering is according to SEQ ID NO: 2; and has at least 60%, at least 65%, at least 70%, at least 75%, at least 80%, at least 85%, at least 90%, at least 95% but less than 100% sequence identity to SEQ ID NOs: 1 or 2.

In one embodiment, the invention relates to a variant of polypeptide of amino acid sequence of SEQ ID NO: 1, wherein the polypeptide has alpha-amylase activity, wherein said variant amino acid sequence comprises a mutation at amino acid position corresponding to positions 202 and/or 186 wherein numbering is according to SEQ ID NO: 2, with the proviso that the mutation in position 186 is not A186G, and wherein the variant has at least 60% sequence identity to SEQ ID Nos: 1 or 2.

In one embodiment, the invention relates to a variant of polypeptide of amino acid sequence comprising a substitution at amino acid position 202 wherein numbering is according to SEQ ID NO: 2, such as L202M. In one embodiment, the polypeptide is a variant amino acid sequence consisting of the substitution L202M wherein numbering is according to SEQ ID NO: 2.

In another embodiment, the invention relates to a variant of polypeptide does not comprise a mutation in position 202 but in at least position 186, which is not the substitution A186D, and potentially other positions, wherein numbering is according to SEQ ID NO: 2.

In another embodiment, the invention relates to a variant of polypeptide of amino acid sequence comprising a substitution at amino acid position 186 wherein numbering is according to SEQ ID NO: 2, such as A186D.

In one embodiment, the invention relates to a variant of polypeptide of SEQ ID NO:1 having a mutation (such as a substitution, deletion, and/or insertion) at one or more positions corresponding to positions 51, 186, 202, 246 and 334, wherein numbering is according to the amino acid sequence set forth in SEQ ID NO: 2. Thus, the variant of the present invention comprises a mutation in at least one of the positions corresponding to positions 202 and/or 186 wherein numbering is according to SEQ ID NO: 2 and may further comprise a mutation in one or more of positions 51, 246 and 334, and in the case wherein the variant comprises a mutation in position 202, then it may also comprise a mutation in position 186 and potentially also any other position, and vice versa. I.e. when the variant comprises a mutation in position 186, then it may also comprise a mutation in position 202 and potentially also any other position. It is to be understood that the variant according to the present invention does not necessarily comprise a mutation in both position 202 and position 186 when it comprises a mutation in other positions.

In one particular embodiment, the polypeptide having alpha-amylase activity has at least 60%, at least 65%, at least 70%, at least 75%, at least 80%, or at least 87%, but less than 100% at least 80%, such as at least 85%, such as at least 90%, such as at least 92%, such as at least 93%, such as at least 94%, such as at least 95%, such as at least 96%, such as at least 97%, such as at least 98%, such as at least 99%, but less than 100% sequence identity to the amino acid sequence of SEQ ID NO: 1 or 2.

Thus, the polypeptide may comprise one or more of the following substitutions relative to the amino acid sequence of SEQ ID NO:2; A51T, A186D, L202M, T246(I/L/V) and S334T.

It will be appreciated by persons skilled in the art that different examples of the polypeptides of the invention will possess a different degree of amino acid sequence identity with the sequence of SEQ ID NO:1. Thus, the polypeptide may comprise or consist of an amino acid sequence which shares at least 85% sequence identity with SEQ ID NO: 1, for example at least 90%, at least 95%, at least 96%, at least 97%, at least 98%, or at least 99% sequence identity to SEQ ID NO: 1. In one embodiment, the number of mutations within the polypeptide relative to the amino acid sequence of SEQ ID NO:1 is between 1 and 20, e.g., between 1 and 10 mutations or between 1 and 5 mutations, such as 1, 2, 3, 4, 5, 6, 7, 8, 9 or 10 mutations.

In one particular subset of the polypeptides having alpha-amylase activity, the polypeptide consists of the amino acid sequence of SEQ ID NO:1 with mutations selected from the group consisting of; A51T+A186D, A51T+L202M, A51T+T2461, A51T+S334T, A186D+L202M, A186D+T2461, A186D+S334T, L202M+T2461, L202M+S334T, T246I+S334T, A51T+A186D+L202M, A51T+A186D+T2461, A51T+A186D+S334T, A51T+L202M+T2461, A51T+L202M+S334T, A51T+T246I+S334T, A186D+L202M+T2461, A186D+L202M+S334T, A186D+T246I+S334T, L202M+T246I+S334T, A51T+A186D+L202M+T2461, A51T+A186D+L202M+S334T, A51T+A186D+T246I+S334T, A51T+L202M+T246I+S334T, A186D+L202M+T246I+S334T, and A51T+A186D+L202M+T246I+S334T, or a fragment thereof having alpha amylase activity.

For example, the polypeptide may comprise or consist of an amino acid sequence of SEQ ID NO:1 with mutations selected from the group consisting of the following:

### (a) L202M;

### (b) A186D;

### (c) L202M+T246+S334T;

### (d) L202M+T246L+S334T;

### (e) A51T+L202M+T246+S334T;

### (f) L202M+T246V; and

### (g) L202M+T246V+S334T;

wherein numbering of amino acid positions is according to the amino acid sequence set forth in SEQ ID NO: 2.

In one embodiment the alpha-amylase is a variant alpha-amylase of a parent alpha-amylase of SEQ ID NO: 19, wherein the variant is selected from the group consisting of: H1*+N54S+V56T+G109A+A174S+G182*+D183*+N195F+V206L+K391A+G476K, H1*+N54S+V56T+R87S+G109A+A174S+G182*+D183*+N195F+V206L+K391A+G476K, H1*+T40G+N54S+V56T+G109A+A174S+G182*+D183*+N195F+V206L+K391A+G476K, H1*+T51K+N54S+V56T+G109A+A174S+G182*+D183*+N195F+V206L+K391A+G476K, H1*+N54S+V56T+K72R+G109A+A174S+G182*+D183*+N195F+V206L+K391A+G476K, H1*+N54S+V56T+K72H+G109A+A174S+G182*+D183*+N195F+V206L+K391A+G476K, H1*+A37H+N54S+V56T+G109A+A174S+G182*+D183*+N195F+V206L+K391A+G476K, H1*+A37M+N54S+V56T+G109A+A174S+G182*+D183*+N195F+V206L+K391A+G476K, H1*+A37V+N54S+V56T+G109A+A174S+G182*+D183*+N195F+V206L+K391A+G476K, H1*+A37S+N54S+V56T+G109A+A174S+G182*+D183*+N195F+V206L+K391A+G476K, H1*+A37Y+N54S+V56T+G109A+A174S+G182*+D183*+N195F+V206L+K391A+G476K, H1*+A37R+N54S+V56T+G109A+A174S+G182*+D183*+N195F+V206L+K391A+G476K, H1*+N54S+V56T+G109A+F113W+A174S+G182*+D183*+N195F+V206L+K391A+G476K, H1*+N54S+V56T+G109A+F113S+A174S+G182*+D183*+N195F+V206L+K391A+G476K, H1*+N54S+V56T+G109A+F113N+A174S+G182*+D183*+N195F+V206L+K391A+G476K, H1*+N54S+V56T+G109A+R116Q+A174S+G182*+D183*+N195F+V206L+K391A+G476K, H1*+N54S+V56T+G109A+R116V+A174S+G182*+D183*+N195F+V206L+K391A+G476K, H1*+N54S+V56T+G109A+R116K+A174S+G182*+D183*+N195F+V206L+K391A+G476K, H1*+N54S+V56T+G109A+Q125P+A174S+G182*+D183*+N195F+V206L+K391A+G476K, H1*+N54S+V56T+G109A+A174S+G182*+D183*+N195F+V206L+S381G+K391A+G476K, H1*+N54S+V56T+G109A+A174S+G182*+D183*+N195F+V206L+M246T+K391A+G476K, H1*+A37L+N54S+V56T+G109A+A174S+G182*+D183*+N195F+V206L+K391A+G476K, H1*+N54S+V56T+G109A+R116A+A174S+G182*+D183*+N195F+V206L+K391A+G476K, H1*+N54S+V56T+G109A+R116H+A174S+G182*+D183*+N195F+V206L+K391A+G476K, H1*+N54S+V56T+G109A+Q125A+A174S+G182*+D183*+N195F+V206L+K391A+G476K, H1*+N54S+V56T+G109A+Q172G+A174S+G182*+D183*+N195F+V206L+K391A+G476K, H1*+N54S+V56T+G109A+Q172R+A174S+G182*+D183*+N195F+V206L+K391A+G476K, H1*+N54S+V56T+G109A+A174S+G182*+D183*+N195F+V206L+D377S+K391A+G476K, H1*+N54S+V56T+G109A+A174S+G182*+D183*+N195F+V206L+D377A+K391A+G476K, H1*+N54S+V56T+G109A+A174S+G182*+D183*+N195F+V206L+S381A+K391A+G476K, H1*+N54S+V56T+G109A+A174S+G182*+D183*+N195F+V206L+G346P+K391A+G476K, H1*+N54S+V56T+G109A+A174S+G182*+D183*+N195F+V206L+1214H+K391A+G476K, H1*+N54S+V56T+G109A+A174S+G182*+D183*+N195F+V206L+1214S+K391A+G476K, H1*+N54S+V56T+G109A+A174S+G182*+D183*+N195F+V206L+K391A+A420Q+G476K, H1*+N54S+V56T+G109A+A174S+G182*+D183*+N195F+V206L+K391A+A420S+G476K, H1*+N54S+V56T+G109A+A174S+G182*+D183*+N195F+V206L+K391A+A420K+G476K, H1*+N54S+V56T+G109A+A174S+G182*+D183*+N195F+V206L+K391A+A420L+G476K, H1*+N54S+V56T+G109A+A174S+G182*+D183*+N195F+V206L+K391A+K391Y+G476K, H1*+N54S+V56T+G109A+A174S+G182*+D183*+N195F+V206L+K391A+P473R+G476K, H1*+N54S+V56T+G109A+A174S+G182*+D183*+N195F+V206L+K391A+P473A+G476K, H1*+N54S+V56T+G109A+A174S+G182*+D183*+N195F+V206L+K391A+P473G+G476K, H1*+N54S+V56T+G109A+A174S+G182*+D183*+N195F+V206L+K391A+T444D+G476K, H1*+N54S+V56T+G109A+A174S+G182*+D183*+N195F+V206L+K391A+T444Y+G476K, H1*+N54S+V56T+G109A+A174S+G182*+D183*+N195F+V206L+S280W+K391A+G476K, H1*+N54S+V56T+G109A+A174S+G182*+D183*+N195F+V206L+S280L+K391A+G476K, H1*+N54S+V56T+K72S+G109A+A174S+G182*+D183*+N195F+V206L+K391A+G476K, H1*+T40K+N54S+V56T+G109A+A174S+G182*+D183*+N195F+V206L+G346T+K391A+G476K, H1*+G50A+N54S+V56T+G109A++Q172N+A174S+G182*+D183*+N195F+V206L+K391A+G476K, H1*+N54S+V56T+G109A+A174S+G182*+D183*+N195F+V206L+K391A+G476K+K484S, H1*K+G50A+N54S+V56T+G109A+W167F+A174S+G182*+D183*+N195F+V206L+K391A+G476K, H1*+N54S+V56T+G109A+L173V+A174S+G182*+D183*+N195F+A204T+V206L+K391A+G476K, H1*+N54S+V56T+G109A+W140Y+A174S+G182*+D183*+N195F+V206L+K391A+G476K, H1*+N54S+V56T+G109A+A174S+G182*+D183*+N195F+V206L+G346T+K391A+G476K+G477A, H1*+N54S+V56T+G109AG255A++A174S+G182*+D183*+N195F+V206L+K391A+G476K, H1*+G50A+N54S+V56T+G109A+F113L+R116L+A174S+G182*+D183*+N195F+V206L+K391A+G476K, H1*+N54S+V56T+G109A+W167F+A174S+G182*+D183*+N195F+V206L+K391A+G476K, H1*+N54S+V56T+K72R+G109A+W167F+A174S+G182*+D183*+N195F+V206L+K391A+G476K, H1*+N54S+V56T+G109A+A174S+G182*+D183*+N195F+V206L+R320V+K391A+G476Y, H1*+N54S+V56T+G109A+R116M+A174S+G182*+D183*+N195F+V206L+K391A+G476K, H1*+N54S+V56T+G109A+Q172Y+A174S+G182*+D183*+N195F+V206L+K391A+G476K, H1*+N54S+V56T+G109A+A174S+G182*+D183*+N195F+V206L+1214L+K391A+G476K, H1*+N54S+V56T+G109A+A174S+G182*+D183*+N195F+V206L+L217V+K391A+G476K, H1*+N54S+V56T+G109A+A174S+G182*+D183*+N195F+V206L+L217H+K391A+G476K, H1*+N54S+V56T+G109A+A174S+G182*+D183*+N195F+V206L+P211 D+K391A+G476K, H1*+N54S+V56T+G109A+A174S+G182*+D183*+N195F+V206L+K391A+K391Q+G476K, H1*+N54S+V56T+G109A+A174S+G182*+D183*+N195F+V206L+K391A+T444H+G476K, H1*+N54S+V56T+G109A+A174S+G182*+D183*+N195F+V206L+K391A+T444V+G476K, H1*+N54S+V56T+G109A+A174S+G182*+D183*+N195F+V206L+K391A+T444R+G476K, H1*+G50A+N54S+V56T+G109A+L173V+A174S+G182*+D183*+N195F+A204V+V206L+K391A+G476K, H1*+G7K+N54S+V56T+G109A+A174S+G182*+D183*+N195F+V206L+R320A+K391A+G476K, H1*+N54S+V56T+G109A+Q172D+A174S+G182*+D183*+N195F+V206L+G346T+K391A+G476K+G477A, H1*+N54S+V56T+G109A+Q118S+A174S+G182*+D183*+N195F+V206L+K391A+G476K, H1*+N54S+V56T+G109A+A174S+G182*+D183*+N195F+V206L+R320A+K391A+G476K, H1*+N54S+V56T+G109A+T134E+A174S+G182*+D183*+N195F+V206L+K391A+G476K, H1*+N54S+V56T+G109A+A174S+G182*+D183*+N195F+V206L+Y382M+K391A+G476K, H1*+N54S+V56T+K72R+G109A+T134E+A174S+G182*+D183*+N195F+V206L+G255A+K391A+G476K, H1*+N54S+V56T+K72R+M105F+G109A+A174S+G182*+D183*+N195F+V206L+G255A+K391A+G476K, H1*+N54S+V56T+M105F+G109A+T134E+A174S+G182*+D183*+N195F+V206L+K391A+G476K, H1*+N54S+V56T+G109A+A174S+G182*+D183*+N195F+V206L+R320A+S323N+D377H+K391A+G476K, H1*+T51E+N54S+V56T+G109A+A174H+G182*+D183*+N195F+V206L+K391A+G476K, H1*+N54S+V56T+G109A+L173A+A174S+G182*+D183*+N195F+V206L+K391A+G476K, H1*+N54S+V56T+G109A+A174S+G182*+D183*+N195F+V206L+Q299V+K391A+G476K, H1*+N54S+V56T+G109A+L173G+A174S+G182*+D183*+N195F+V206L+K391A+G476K, H1*+N54S+V56T+G109A+A174S+G182*+D183*+N195F+V206L+T355L+K391A+G476K, H1*+G50A+N54S+V56T+G109A+A174S+G182*+D183*+N195F+A204T+V206L+K391A+G476K, H1*+N54S+V56T+K72R+G109A+A174S+G182*+D183*+N195F+V206L+R320A+S323N+K391A+G476K, H1*+N54S+V56T+G109A+A174S+G182*+D183*+N195F+V206L+M246L+K391A+G476K, H1*+N54S+V56T+K72Q+G109A+W167H+A174S+G182*+D183*+N195F+V206L+K391A+G476K, H1*+N54S+V56T+K72R+G109A+T134E+A174S+G182*+D183*+N195F+V206L+K391A+G476K, H1*+N54S+V56T+K72R+G109A+T134E+A174S+G182*+D183*+N195F+V206L+D377H+K391A+G476K, H1*+N54S+V56T+K72R+G109A+T134E+A174S+G182*+D183*+G184T+N195F+V206L+K391A+G476K, H1*+N54S+V56T+G109A+Q118R+T134E+A174S+G182*+D183*+N195F+V206L+G255A+K391A+G476K, H1*+N54S+V56T+G109A+Q118R+A174S+G182*+D183*+N195F+V206L+D377H+K391A+G476K, H1*+N54S+V56T+G109A+T134E+A174S+G182*+D183*+G184T+N195F+V206L+D377H+K391A+G476K, H1*+N54S+V56T+G109A+Q172D+A174Q+G182*+D183*+N195F+V206L+K391A+G476K+G477A, H1*+N54S+V56T+K72R+G109A+A174S+G182*+D183*+G184T+N195F+V206L+G255A+K391A+G476K, H1*+N54S+V56T+G109A+A174S+G182*+D183*+G184T+N195F+V206L+D377H+K391A+G476K, H1*+N54S+V56T+G109A+A174S+G182*+D183*+N195F+V206L+G255A+D377H+K391A+G476K, H1*+N54S+V56T+G109A+T134E+A174S+G182*+D183*+G184T+N195F+V206L+K391A+G476K, H1*+N54S+V56T+G109A+A174S+G182*+D183*+N195F+V206L+V238T+D377H+K391A+G476K, H1*+N54S+V56T+G109A+A174S+G182*+D183*+N195F+V206L+V291A+D377H+K391A+G476K, H1*+N54S+V56T+K72R+G109A+T134E+A174S+G182*+D183*+G184T+N195F+V206L+G255A+K391A+G476K, H1*+N54S+V56T+G109A+L173H+A174S+G182*+D183*+N195F+V206L+K391A+G476K, H1*+N54S+V56T+K72N+G109A+A174S+G182*+D183*+N195F+V206L+K391A+G476K, H1*+N54S+V56T+K72N+G109A+W167L+A174S+G182*+D183*+N195F+V206L+K391A+G476K, H1*+N54S+V56T+G109A+Q118R+T134E+A174S+G182*+D183*+N195F+V206L+G255A+D377H+K391A+G476K, H1*+N54S+V56T+K72R+G109A+A174S+G182*+D183*+N195F+G184T+V206L+K391A+G476K, H1*+N54S+V56T+K72R+G109A+Q118R+A174S+G182*+D183*+N195F+V206L+G255A+D377H+K391A+G476K, H1*+N54S+V56T+G109A+G182*+D183*+N195F+V206L+D377H+K391A+G476K, H1*+N54S+V56T+G109A+A174S+G182*+D183*+N195F+D377H+K391A+G476K, H1*+N54S+V56T+G109A+A174S+G182*+D183*+N195F+V206L+D377H+G476K, H1*+N54S+V56T+G109A+A174S+G182*+D183*+N195F+V206L+D377H+K391A, H1*+N54S+V56T+G109A+A174S+G182*+D183*+N195F+V206L+D377S+K391A+G476K, H1*+N54S+V56T+G109A+R116E+A174S+G182*+D183*+N195F+V206L+K391A+G476K, H1*+N54S+V56T+G109A+T134E+A174S+G182*+D183*+N195F+V206L+D377H+K391A+G476K, H1*+N54S+V56T+G109A+R116S+A174S+G182*+D183*+N195F+V206L+K391A+G476K, H1*+N54S+V56T+G109A+R1161+A174S+G182*+D183*+N195F+V206L+K391A+G476K, H1*+N54S+V56T+G109A+R116G+A174S+G182*+D183*+N195F+V206L+K391A+G476K, H1*+T51A+N54S+V56T+G109A+Q172S+A174S+G182*+D183*+N195F+V206L+K391A+G476K, H1*+T51E+N54S+V56T+G109A+Q172T+A174S+G182*+D183*+N195F+V206L+K391A+G476K, H1*+T51S+N54S+V56T+G109A+Q172N+A174S+G182*+D183*+N195F+V206L+K391A+G476K, H1*+T51E+N54S+V56T+G109A+Q172L+A174S+G182*+D183*+N195F+V206L+K391A+G476K, H1*+T51E+N54S+V56T+G109A+Q172R+A174S+G182*+D183*+N195F+V206L+K391A+G476K, H1*+N54S+V56T+G109A+Q172K+A174S+G182*+D183*+N195F+V206L+K391A+G476K, H1*+N54S+V56T+K72R+G109A+Q172S+A174S+G182*+D183*+N195F+V206L+K391A+G476K, H1*+N54S+V56T+K72H+G109A+Q172T+A174S+G182*+D183*+N195F+V206L+K391A+G476K, H1*+N54S+V56T+K72Q+G109A+Q172R+A174S+G182*+D183*+N195F+V206L+K391A+G476K, H1*+N54S+V56T+K72R+G109A+Q172T+A174S+G182*+D183*+N195F+V206L+K391A+G476K, H1*+N54S+V56T+K72M+G109A+Q172S+A174S+G182*+D183*+N195F+V206L+K391A+G476K, H1*+N54S+V56T+K72Q+G109A+Q172M+A174S+G182*+D183*+N195F+V206L+K391A+G476K H1*+N54S+V56T+K72S+G109A+A174H+G182*+D183*+N195F+V206L+K391A+G476K, H1*+N54S+V56T+K72Q+G109A+A174D+G182*+D183*+N195F+V206L+K391A+G476K, H1*+N54S+V56T+K72R+G109A+A174D+G182*+D183*+N195F+V206L+K391A+G476K, H1*+N54S+V56T+K72Q+G109A+A174K+G182*+D183*+N195F+V206L+K391A+G476K, H1*+N54S+V56T+G109A+A174G+G182*+D183*+N195F+V206L+K391A+G476K, H1*+N54S+V56T+K72R+G109A+A174Q+G182*+D183*+N195F+V206L+K391A+G476K, H1*+N54S+V56T+K72Q+G109A+A174G+G182*+D183*+N195F+V206L+K391A+G476K, H1*+N54S+V56T+K72S+G109A+A174N+G182*+D183*+N195F+V206L+K391A+G476K, H1*+N54S+V56T+K72R+G109A+A174H+G182*+D183*+N195F+V206L+K391A+G476K, H1*+G50A+N54S+V56T+G109A+A174E+G182*+D183*+N195F+A204T+V206L+S323N+K391A+G476K, H1*+N54S+V56T+G109A+A174S+G182*+D183*+N195F+A204G+V206L+K391A+G476K, H1*+N54S+V56T+G109A+A174S+G182*+D183*+N195F+V206L+Y3821+K391A+G476K, H1*+N54S+V56T+G109A+A174S+G182*+D183*+N195F+V206L+1214W+K391A+G476K, H1*+N54S+V56T+G109A+A174S+G182*+D183*+N195F+A204S+V206L+D377H+K391A+G476K, H1*+N54S+V56T+G109A+A174S+G182*+D183*+N195F+V206L+T334S+D377H+K391A+G476K, H1*+N54S+V56T+G109A+A174S+G182*+D183*+N195F+V206L+A263G+D377H+K391A+G476K, H1*+N54S+V56T+G109A+A174S+G182*+D183*+N195F+V206L+M286F+D377H+K391A+G476K, H1*+N54S+V56T+G109A+A174S+G182*+D183*+N195F+V206L+L235V+D377H+K391A+G476K, H1*+N54S+V56T+M105L+G109A+A174S+G182*+D183*+N195F+V206L+D377H+K391A+G476K, H1*+N54S+V56T+M1051+G109A+A174S+G182*+D183*+N195F+V206L+D377H+K391A+G476K, H1*+N54S+V56T+M105V+G109A+A174S+G182*+D183*+N195F+V206L+D377H+K391A+G476K, H1*+N54S+V56T+G109A+A174S+G182*+D183*+N195F+V206L+M2461+D377H+K391A+G476K, H1*+N54S+V56T+G109A+A174S+G182*+D183*+N195F+V206L+L2501+D377H+K391A+G476K, H1*+N54S+V56T+G109A+A174S+G182*+D183*+N195F+V206L+L250V+D377H+K391A+G476K, H1*+N54S+V56T+G109A+A174S+G182*+D183*+N195F+V206L+K391Y+G476K, H1*+N54S+V56T+G109A+A174S+G182*+D183*+N195F+V206L+K391V+G476K, H1*+N54S+V56T+G109A+A174S+G182*+D183*+N195F+V206L+K391M+G476K, H1*+N54S+V56T+G109A+T165G+A174S+G182*+D183*+N195F+V206L+D377H+K391A+G476K, H1*+N54S+V56T+G109S+A174S+G182*+D183*+N195F+V206L+K391A+G476K, H1*+N54S+V56T+G109A+A174V+G182*+D183*+N195F+V206L+K391A+G476K, H1*+N54S+V56T+G109A+A174S+G182*+D183*+N195F+V206L+M246F+D377H+K391A+G476K, H1*+N54S+V56T+G109A+A174S+G182*+D183*+N195F+V206L+M246V+D377H+K391A+G476K, H1*+N54S+V56T+G109A+A174S+G182*+D183*+N195F+V206L+M246S+D377H+K391A+G476K, H1*+N54S+V56T+G109A+A174S+G182*+D183*+N195F+V206L+K391R+G476K, H1*+N54S+V56T+G109A+A174S+G182*+D183*+N195F+V206L+K391H+G476K, H1*+N54S+V56T+G109A+A174S+G182*+D183*+N195F+V206L+K391W+G476K, H1*+N54S+V56T+G109A+A174S+G182*+D183*+N195F+V206L+K391 I+G476K, H1*+N54S+V56T+G109A+A174S+G182*+D183*+N195F+V206L+L250T+D377H+K391A+G476K, H1*+N54S+V56T+G109A+A174S+G182*+D183*+N195F+V206L+L250A+D377H+K391A+G476K, H1*+N54S+V56T+G109A+A174S+G182*+D183*+N195F+V206L+L250F+D377H+K391A+G476K, H1*+N54S+V56T+G109A+A174S+G182*+D183*+N195F+V206L+L250M+D377H+K391A+G476K, H1*+N54S+V56T+G109A+Q172H+L173A+A174P+G182*+D183*+N195F+V206L+K391A+G476K, H1*+N54S+V56T+G109A+A174S+G182*+D183*+G184T+N195F+V206L+G255A+D377H+K391A+G476K, H1*+N54S+V56T+G109A+A174S+G182*+D183*+N195F+V206L+S376T+K391A+G476K, H1*+N54S+V56T+G109A+A174S+G182*+D183*+N195F+V206L+S376V+K391A+G476K, H1*+N54S+V56T+G109A+Q172E+L173+A174N+G182*+D183*+N195F+V206L+K391A+G476K, H1*+N54S+V56T+G109A+Q172D+L173A+A174T+G182*+D183*+N195F+V206L+K391A+G476K, H1*+N54S+V56T+G109A+A174S+G182*+D183*+N195F+V206L+Y267M+D377H+K391A+G476K, H1*+N54S+V56T+G109A+A174S+G182*+D183*+N195F+V206L+V238G+K391A+G476K, H1*+N54S+V56T+M105V+G109A+A174S+G182*+D183*+N195F+V206L+K391A+G476K, H1*+N54S+V56T+G109A+A174S+G182*+D183*+N195F+V206L+V264T+K391A+G476K, H1*+N54S+V56T+G109A+A174S+G182*+D183*+N195F+V206L+V2641+K391A+G476K, H1*+N54S+V56T+G109A+W167H+Q172N+L173A+A174S+G182*+D183*+N195F+V206L+K391A+G476K, H1*+N54S+V56T+G109A+Q172N+L173T+A174S+G182*+D183*+N195F+V206L+K391A+G476K, H1*+N54S+V56T+G109A+Q172E+L173M+A174H+G182*+D183*+N195F+V206L+K391A+G476K, H1*+N54S+V56T+G109A+W167Y+Q172E+L173V+A174H+G182*+D183*+N195F+V206L+K391A+G476K, H1*+N54S+V56T+G109A+L173A+A174T+G182*+D183*+N195F+V206L+K391A+G476K, H1*+N54S+V56T+G109A+A174S+G182*+D183*+N195F+V206L+V291A+F328L+K391A+G476K, H1*+N54S+V56T+G109A+A174S+G182*+D183*+N195F+V206L+M246L+K391A+G476K, H1*+N54S+V56T+G109A+A174S+G182*+D183*+N195F+V206L+L250V+K391A+G476K, H1*+N54S+V56T+G109A+A174S+G182*+D183*+N195F+V206L+L250F+K391A+G476K, H1*+N54S+V56T+G109A+A174S+G182*+D183*+N195F+V206L+L250M+K391A+G476K, H1*+N54S+V56T+G109A+T165G+A174S+G182*+D183*+N195F+V206L+K391A+G476K, H1*+T51A+N54S+V56T+G109A+Q172R+A174S+G182*+D183*+N195F+V206L+K391A+G476K, H1*+N54S+V56T+G109A+A174S+G182*+D183*+N195F+V206L+K391 L+G476K, H1*+N54S+V56T+G109A+F113Q+R116H+Q172G+A174S+G182*+D183*+N195F+V206L+K391A+G476K, H1*+N54S+V56T+G109S+F113Q+R116Q+A174S+G182*+D183*+N195F+V206L+K391A+G476K, H1*+N54S+V56T+G109A+Q172D+A174S+G182*+D183*+N195F+A204G+V206L+K391A+P473R+G476K, H1*+N54S+V56T+K72R+G109A+T134E+A174S+G182*+D183*+N195F+A204G+V206L+G255A+K391A+P473R+G476K, H1*+N54S+V56T+K72R+G109A+W167F+A174S+G182*+D183*+N195F+A204G+V206L+K391A+G476K, H1*+N54S+V56T+K72R+G109A+A174S+G182*+D183*+G184T+N195F+A204G+V206L+K391A+P473R+G476K, H1*+N54S+V56T+G109A+W167F+Q172N+A174S+G182*+D183*+N195F+V206L+K391A+P473R+G476K, H1*+N54S+V56T+G109A+W167F+Q172G+A174S+G182*+D183*+N195F+V206L+K391A+P473G+G476K, H1*+N54S+V56T+G109A+W167F+Q172G+A174S+G182*+D183*+N195F+V206L+K391A+P473R+G476K, H1*+N54S+V56T+K72R+G109A+W167F+Q172R+A174S+G182*+D183*+N195F+V206L+K391A+G476K, H1*+N54S+V56T+K72R+G109A+R116H+W167F+Q172R+A174S+G182*+D183*+N195F+V206L+K391A+G476K, H1*+N54S+V56T+M105F+G109A+A174S+G182*+D183*+N195F+V206L+R320A+S323N+K391A+C474V+G476K, H1*+N54S+V56T+K72R+G109A+A174S+G182*+D183*+N195F+V206L+G255A+K391A+C474V+G476K, H1*+N54S+V56T+G109A+T134E+A174S+G182*+D183*+N195F+V206L+K391A+C474V+G476K, H1*+N54S+V56T+G109A+A174S+G182*+D183*+N195F+V206L+D377H+K391A+C474V+G476K, H1*+N54S+V56T+K72R+G109A+A174S+G182*+D183*+N195F+V206L+K391A+C474V+G476K, H1*+N54S+V56T+G109A+A174S+G182*+D183*+N195F+V206L+A265G+K391A+T444Q+G476K+G477A, H1*+A37V+N54S+V56T+G109A+W167F+Q172G+A174S+G182*+D183*+N195F+V206L+K391A+G346T+G477A+G476K, H1*+N54S+V56T+G109A+R116Q+Q172D+A174S+G182*+D183*+N195F+V206L+G346T+K391A+T444Q+G477A+G476K, H1*+N54S+V56T+G109A+R116Q+W167F+Q172N+A174S+G182*+D183*+N195F+V206L+G346T+K391A+T444Q+G477A+G476K, H1*+N54S+V56T+G109S+R116Q+W167F+Q172N+A174S+G182*+D183*+N195F+V206L+K391A+G476K, H1*+N54S+V56T+G109S+W167F+Q172G+A174S+G182*+D183*+N195F+V206L+K391A+G476K, H1*+N54S+V56T+G109A+F113Q+Q172G+A174S+G182*+D183*+N195F+V206L+K391A+T444Q+G476K H1*+N54S+V56T+G109A+F113Q+R116H+Q172G+A174S+G182*+D183*+N195F+V206L+K391A+T444Q+G476K, H1*+N54S+V56T+G109A+F113Q+A174S+G182*+D183*+N195F+V206L+K391A+C474V+G476K, H1*+N54S+V56T+G109A+Q172N+A174S+G182*+D183*+N195F+V206L+V2641+K391A+C474V+G476K, H1*+N54S+V56T+G109A+Q172N+A174S+G182*+D183*+N195F+V206L+K391A+C474V+G476K, H1*+N54S+V56T+G109A+A174S+G182*+D183*+N195F+V206L+S280Q+H321Y+K391A+C474V+G476K, H1*+N54S+V56T+K72R+G109A+T134E+A174S+G182*+D183*+N195F+V206L+G255A+K391A+C474V+G476K, H1*+N54S+V56T+G109A+F113Q+R116H+Q172G+A174S+G182*+D183*+N195F+V206L+K391A+T444Q+P473R+G476K, H1*+N54S+V56T+G109A+F113Q+R116Q+Q172G+A174S+G182*+D183*+N195F+V206L+K391A+T444Q+G476K, H1*+N54S+V56T+G109A+R116H+W167F+Q172N+A174S+G182*+D183*+N195F+V206L+K391A+G476K, H1*+N54S+V56T+G109A+F113Q+R116H+T165G+Q172G+A174S+G182*+D183*+N195F+V206L+K391A+T444Q+G476K, H1*+N54S+V56T+G109A+F113Q+R116Q+T165G+Q172G+A174S+G182*+D183*+N195F+V206L+K391A+T444Q+G476K, H1*+N54S+V56T+G109A+F113Q+R116H+W167F+Q172R+A174S+G182*+D183*+N195F+V206L+F2891+K391A+G476K, H1*+N54S+V56T+G109A+F113Q+Q172N+A174S+G182*+D183*+N195F+V206L+K391A+T444Q+G476K, H1*+N54S+V56T+G109A+F113Q+R116H+Q172N+A174S+G182*+D183*+N195F+V206L+K391A+G476K, H1*+N54S+V56T+G109A+F113Q+R116Q+Q172M+A174S+G182*+D183*+N195F+V206L+K391A+T444Q+G476K, H1*+N54S+V56T+G109A+F113Q+R116H+Q172M+A174S+G182*+D183*+N195F+V206L+K391A+G476K, H1*+N54S+V56T+G109A+R116H+Q172M+A174S+G182*+D183*+N195F+V206L+K391A+G476K, H1*+N54S+V56T+G109A+R116Q+A174S+G182*+D183*+N195F+V206L+A265G+K391A+T444Q+P473R+G476K, H1*+N54S+V56T+G109A+R116Q+A174S+G182*+D183*+N195F+V206L+K391A+T444Q+P473R+G476K, H1*+N54S+V56T+K72R+G109A+T134E+T165G+A174S+G182*+D183*+N195F+V206L+G255A+K391A+G476K, H1*+N54S+V56T+G109A+F113Q+R116H+W167F+Q172R+A174S+G182*+D183*+N195F+V206L+K391A+G476K, H1*+N54S+V56T+G109A+R116Q+W167F+Q172R+A174S+G182*+D183*+N195F+V206L+A265G+K391A+P473R+G476K, H1*+N54S+V56T+G109A+F113Q+A174S+G182*+D183*+N195F+V206L+K391A+G476K, H1*+N54S+V56T+G109A+Q172N+A174S+G182*+D183*+N195F+V206L+K391A+G476K, H1*+T51V+N54S+V56T+G109A+Q172D+A174S+G182*+D183*+N195F+V206L+K391A+G476K, H1*+N54S+V56T+G109A+A174S+G182*+D183*+N195F+V206L+S280Q+H321Y+K391A+G476K, H1*+N54S+V56T+G109A+F113Q+R116H+Q172N+A174S+G182*+D183*+N195F+V206L+K391A+G476K, H1*+N54S+V56T+G109A+F113Q+R116H+Q172G+L173V+A174S+G182*+D183*+N195F+V206L+K391A+T444Q+P473R+G476K, H1*+N54S+V56T+G109A+R116Q+W167F+Q172N+L173V+A174S+G182*+D183*+N195F+V206L+K391A+T444Q+G476K, H1*+N54S+V56T+G109A+G133Q+W167F+Q172N+A174S+G182*+D183*+N195F+V206L+Q395P+K391A+G476K, H1*+N54S+V56T+G109A+F113Q+R116H+W167F+Q172N+L173V+A174S+G182*+D183*+N195F+V206L+K391A+G476K, H1*+A37V+N54S+V56T+K72R+G109A+R116H+W167F+Q172R+A174S+G182*+D183*+G184 T+N195F+V206L+K391A+P473R+G476K, H1*+N54S+V56T+K72R+G109A+F113Q+R116H+W167F+Q172G+L173V+A174S+G182*+D183*+G184T+N195F+V206L+K391A+T444Q+P473R+G476K, H1*+N54S+V56T+G109S+R116Q+W167F+Q172R+A174S+G182*+D183*+N195F+V206L+K391A+G476K, H1*+N54S+V56T+G109S+R116Q+W167F+Q172G+A174S+G182*+D183*+N195F+V206L+K391A+P473R+G476K, H1*+N54S+V56T+G109S+F113Q+W167F+Q172N+A174S+G182*+D183*+N195F+V206L+K391A+G476K, H1*+A37V+N54S+V56T+G109S+F113Q+W167F+Q172R+A174S+G182*+D183*+N195F+V206L+K391A+G476K, H1*+N54S+V56T+K72R+G109A+R116H+T134E+W167F+Q172G+L173V+A174S+G182*+D183*+N195F+V206L+G255A+K391A+G476K, H1*+N54S+V56T+K72R+G109A+R116H+T134E+W167F+Q172G+L173V+A174S+G182*+D183*+N195F+V206L+G255A+K391A+Q395P+T444Q+P473R+G476K, H1*+N54S+V56T+K72R+G109A+F113Q+R116Q+W167F+Q172G+A174S+G182*+D183*+G184T+N195F+V206L+K391A+P473R+G476K, H1*+N54S+V56T+K72R+G109A+F113Q+R116Q+T134E+W167F+Q172R+A174S+G182*+D183*+N195F+V206L+G255A+K391A+T444Q+P473R+G476K, H1*+N54S+V56T+K72R+G109A+F113Q+R116H+T134E+W167F+Q172R+A174S+G182*+D183*+N195F+V206L+G255A+K391A+Q395P+G476K, H1*+N54S+V56T+K72R+G109A+T134E+W167F+Q172N+A174S+G182*+D183*+N195F+V206 L+G255A+A265G+K391A+T444Q+P473R+G476K, H1*+N54S+V56T+K72R+G109A+G133Q+W167F+Q172R+A174S+G182*+D183*+N195F+V206L+K391A+G476K, H1*+N54S+V56T+K72R+G109A+F113Q+R116Q+W167F+Q172G+A174S+G182*+D183*+N195F+A204G+V206L+K391A+G476K, H1*+N54S+V56T+K72R+G109A+F113Q+R116Q+W167F+Q172N+L173V+A174S+G182*+D183*+G184T+N195F+V206L+K391A+T444Q+P473R+G476K, H1*+A37H+N54S+V56T+G109A+F113Q+W167F+Q172N+A174S+G182*+D183*+N195F+V206L+K391A+G476K, H1*+N54S+V56T+G109A+F113Q+W167F+Q172N+A174S+G182*+D183*+N195F+V206L+K391A+T459N+G476K, H1*+N54S+V56T+G109A+F113Q+R116Q+A174S+G182*+D183*+N195F+V206L+K391A+P473R+G476K, H1*+N54S+V56T+A60V+G109A+R116Q+W167F+Q172N+A174S+G182*+D183*+N195F+V206L+K391A+G476K, H1*+N54S+V56T+A60V+G109A+R116H+Q172R+L173V+A174S+G182*+D183*+N195F+V206 L+K391A+G476K, H1*+A37V+N54S+V56T+A60V+G109A+R116Q+T165G+A174S+G182*+D183*+N195F+V206L+K391A+G476K, H1*+A37H+N54S+V56T+A60V+G109A+R116Q+T165G+A174S+G182*+D183*+N195F+V206L+K391A+G476K, H1*+N54S+V56T+A60V+G109A+R116Q+W167F+Q172G+L173V+A174S+G182*+D183*+N195F+V206L+K391A+G476K, H1*+N54S+V56T+A60V+G109A+R116Q+A174S+G182*+D183*+N195F+V206L+K391A+G476K, H1*+A37H+N54S+V56T+A60V+G109A+R116Q+W167F+Q172R+A174S+G182*+D183*+N195F+V206L+K391A+G476K, H1*+A37H+N54S+V56T+A60V+G109A+R116Q+W167F+Q172N+A174S+G182*+D183*+N195F+V206L+K391A+G476K, H1*+N54S+V56T+A60V+G109A+R116Q+T165G+A174S+G182*+D183*+N195F+V206L+K391A+T444Q+P473R+G476K, H1*+N54S+V56T+A60V+G109A+R116Q+A174S+G182*+D183*+N195F+V206L+K391A+T444 Q+P473R+G476K, H1*+N54S+V56T+A60V+G109A+R116Q+W167F+Q172N+L173V+A174S+G182*+D183*+N195F+V206L+K391A+G476K, H1*+N54S+V56T+A60V+G109A+R116Q+W167F+Q172R+A174S+G182*+D183*+N195F+V206L+K391A+G476K, H1*+N54S+V56T+G109A+R116A+W167F+Q172R+A174S+G182*+D183*+N195F+A204G+V206L+K391A+P473R+G476K, H1*+N54S+V56T+G109A+R116A+A174S+G182*+D183*+N195F+A204G+V206L+K391A+P473R+G476K, H1*+N54S+V56T+G109A+R116A+T165G+A174S+G182*+D183*+N195F+V206L+K391A+T444Q+P473R+G476K, H1*+N54S+V56T+G109A+G182*+D183*+N195F+K391A+G476K, H1*+N54S+V56T+G109A+G182*+D183*+N195F+V206L, N54S+V56T+G109A+G182*+D183*+N195F, N54S+V56T+G109A+A174S+G182*+D183*+N195F+V206L+K391A+G476K, H1*+N54S+V56T+G109A+G182*+D183*+N195F+V206L+K391A+G476K, H1*+N54S+V56T+G109A+A174S+G182*+D183*+N195F+V206L+K391A, H1*+N54S+V56T+G109A+A174S+G182*+D183*+N195F+G476K, H1*+N54S+V56T+A174S+G182*+D183*+N195F+V206L+G273R+K391A+G476K, H1*+N54S+V56T+G109A+A174S+G182*+D183*+N195F+V206L, N54S+V56T+G109A+A174S+G182*+D183*+N195F+K391A+G476K, H1*+N54S+V56T+G109A+F113Q+Q172M+A174S+G182*+D183*+N195F+V206L+K391A+G476K, H1*+N54S+V56T+K72R+G109A+F113Q+T134E+A174S+G182*+D183*+N195F+V206L+G255A+K391A+G476K, H1*+N54S+V56T+K72R+G109A+F113Q+T134E+A174S+G182*+D183*+N195F+V206L+G255A+K391A+P473R+G476K, H1*+N54S+V56T+K72R+G109A+R116Q+A174S+G182*+D183*+G184T+N195F+V206L+K391A+G476K, H1*+A37V+N54S+V56T+G109A+R116A+T165G+A174S+G182*+D183*+N195F+V206L+K391A+G476K, H1*+A37V+N54S+V56T+G109A+R116Q+T165G+A174S+G182*+D183*+N195F+V206L+M246F+K391A+G476K, H1*+V56A+G109A+A174S+G182*+D183*+N195F+V206L+G476K, H1*+N54S+V56T+K72R+G109A+F113Q+R116H+A174S+G182*+D183*+G184T+N195F+V206 L+K391A+T444Q+G476K, H1*+N54S+V56T+G109S+F113Q+A174S+G182*+D183*+N195F+V206L+K391A+T444Q+G476K, H1*+N54S+V56T+G109A+F113Q+R116H+W167F+Q172G+L173V+A174S+G182*+D183*+N195F+V206L+K391A+P473R+G476K, H1*+N54S+V56T+G109A+F113Q+R116Q+Q172N+A174S+G182*+D183*+N195F+V206L+A265G+K391A+P473R+G476K, H1*+N54S+V56T+G109A+F113Q+W167F+Q172N+A174S+G182*+D183*+N195F+A204G+V206L+K391A+P473R+G476K, H1*+N54S+V56T+G109A+F113Q+R116H+W167F+Q172R+A174S+G182*+D183*+N195F+A204G+V206L+K391A+P473R+G476K, H1*+N54S+V56T+G109A+R116A+A174S+G182*+D183*+N195F+V206L+K391A+T444Q+P473R+G476K, H1*+N54S+V56T+G109A+R116H+Q172R+A174S+G182*+D183*+N195F+A204G+V206L+K391A+P473R+G476K, H1*+A37V+N54S+V56T+G109A+R116Q+T165G+A174S+G182*+D183*+N195F+V206L+K391A+G476K, H1*+A37H+N54S+V56T+G109A+R116H+T165G+A174S+G182*+D183*+N195F+V206L+M246F+K391A+G476K, H1*+N54S+V56T+G109A+A174S+G182*+D183*+N195F+K391A+G476K, H1*+N54S+V56T+G109A+G133S+A174S+G182*+D183*+N195F+K391A+G476H, H1*+N54S+V56T+G109A+R116A+Q172N+L173V+A174S+G182*+D183*+N195F+V206L+K391A+G476K, H1*+A37V+N54S+V56T+G109A+R116H+T165G+A174S+G182*+D183*+N195F+V206L+K391A+G476K, H1*+P45A+N54S+V56T+G109A+A174S+G182*+D183*+N195F+V206L+K391A+C474V+G476K, H1*+N54S+V56T+G109A+A174S+G182*+D183*+N195F+V206L+P364A+K391A+C474V+G476K, H1*+N54S+V56T+G109A+Q118R+T134E+A174S+G182*+D183*+N195F+V206L+K391A+G476K, H1*+N54S+V56T+G109A+A174S+G182*+D183*+G184T+N195F+V206L+K391A+G476K, H1*+G50A+T51A+N54S+V56T+G109A+A174S+G182*+D183*+N195F+V206L+K391A+G476K, H1*+N54S+V56T+G109A+A174S+G182*+D183*+N195F+V206L+V264F+K391A+G476K, H1*+N54S+V56T+G109A+A174S+G182*+D183*+N195F+V206L+Y382L+K391A+G476K, H1*+G7K+N54S+V56T+G109A+A174S+G182*+D183*+N195F+V206L+K391A+G476K, H1*+N54S+V56T+G109A+A174S+G182*+D183*+N195F+V206L+G255A+Q256A+K391A+G476K, H1*+N54S+V56T+G109A+W167Y+A174S+G182*+D183*+N195F+V206L+K391A+G476K, H1*+N54S+V56T+G109A+W167H+A174S+G182*+D183*+N195F+V206L+K391A+G476K, H1*+N54S+V56T+G109A+T165V+A174S+G182*+D183*+N195F+V206L+K391A+G476K, H1*+N54S+V56T+G109A+A174S+G182*+D183*+N195F+V206L+K391A+G476K, H1*+N54S+V56T+G109A+A174N+G182*+D183*+N195F+V206L+K391A+G476K, H1*+N54S+V56T+G109A+A174Q+G182*+D183*+N195F+V206L+K391A+G476K, H1*+N16H+V17L+N54S+V56T+G109A+A174S+G182*+D183*+N195F+V206L+Y382F+K391A+G476K, H1*+Q32S+N54S+V56T+G109A+A174S+G182*+D183*+N195F+V206L+Q385L+K391A+G476K, H1*+N54S+V56T+R87S+G109A+R171H+A174S+G182*+D183*+N195F+V206L+K391A+G476K, H1*+N54S+V56T+G109A+R116H+Q169E+Q172K+A174*+G182*+D183*+N195F+V206L+K391A+G476K, H1*+N54S+V56T+G109A+A174*+G182*+D183*+N195F+V206L+K391A+G476K, H1*+K72R+G109A+F113Q+R116Q+W167F+Q172G+A174S+G182*+D183*+G184T+N195F+V206L+K391A+P473R+G476K, H1*+N54S+V56T+K72R+G109A+F113Q+R116Q+W167F+Q172G+A174S+G182*+D183*+G184T+N195F+V206L+P473R+G476K, H1*+N54S+V56T+G109A+Q169E+Q172G+A174*+G182*+D183*+N195F+V206L+K391A+G476K, H1*+N54S+V56T+K72R+G109A+F113Q+R116Q+W167F+Q172G+A174S+G182*+D183*+G184T+N195F+K391A+P473R+G476K, H1*+N54S+V56T+G109A+R116W+Q169E+Q172K+A174*+G182*+D183*+N195F+V206L+K391A+G476K, H1*+N54S+V56T+G109A+Q169E+Q172K+A174*+G182*+D183*+N195F+V206F+K391A+G476K, H1*+V56T+G109A+A174S+G182*+D183*+N195F+V206L+K391A+G476K, H1*+N54S+V56T+G109A+A174S+G182*+D183*+N195F+V206L+N260G+K391A+G476K, H1*+N54S+V56T+K72R+G109A+A174S+G182*+D183*+N195F+V206L+K391A+G476K, H1*+N54S+V56T+G109A+Q169E+Q172K+A174*+G182*+D183*+G184T+N195F+V206L+K391A+G476K, H1*+N54S+V56T+G109A+Q169E+Q172K+A174*+G182*+D183*+N195F+V206L+Y295N+K391A+G476K, H1*+N54S+V56T+G109A+W167F+A174S+G182*+D183*+N195F+V206L+K391A+G476K, H1*+N54S+V56T+G109A+Q169E+Q172K+A174*+G182*+D183*+A186N+N195F+V206L+K391A+G476K, H1*+N54S+V56T+G109A+Q169E+Q172K+A174*+G182*+D183*+N195F+V206L+A288V+K391A+G476K, H1*+N54S+V56T+G109A+Q169E+Q172K+A174*+G182*+D183*+N195F+V206L+G255A+K391A+G476K, H1*+N54S+G109A+Q169E+Q172K+A174*+G182*+D183*+N195F+V206L+K391A+G476K, H1*+W48F+N54S+V56T+G109A+Q169E+Q172K+A174*+G182*+D183*+N195F+V206L+K391A+G476K, H1*+N54S+V56T+G109A+Q169E+Q172K+A174*+G182*+D183*+N195F+V206L+V291A+K391A+G476K, H1*+N54S+V56T+G109A+F113Q+A174S+G182*+D183*+N195F+V206L+K391A+G476K, H1*+N54S+V56T+G109A+Q169E+Q172K+A174*+G182*+D183*+N195F+V206L+R320A+S323N+K391A+G476K, H1*+V56T+G109A+Q169E+Q172K+A174*+G182*+D183*+N195F+V206L+K391A+G476K, H1*+N54S+V56T+K72R+G109A+F113Q+R116H+W167F+Q172G+A174S+G182*+D183*+G184T+N195F+V206L+K391A+P473R+G476K, H1*+N54S+V56T+G109A+A174S+G182*+D183*+N195F+V206L+N270T+K391A+G476K, H1*+N54S+V56T+G109A+Q169E+Q172K+A174*+G182*+D183*+N195F+V206L+G476K, H1*+N54S+V56T+G109A+Q169E+Q172K+A174*+G182*+D183*+N195F+V206L+N260G+K391A+G476K, H1*+N54S+V56T+G109A+F113Q+R116H+A174S+G182*+D183*+N195F+V206L+K391A+G476K, H1*+N54S+V56T+G109A+Q172G+A174S+G182*+D183*+N195F+V206L+K391A+G476K, H1*+N54S+V56T+G109A+A174S+A186D+G182*+D183*+N195F+V206L+K391A+G476K, H1*+N54S+V56T+G109A+Q169E+Q172K+A174*+G182*+D183*+N195F+V206L+A225V+K391A+G476K, H1*+G109A+Q169E+Q172K+A174*+G182*+D183*+N195F+V206L+K391A+G476K, H1*+N54S+V56T+G109A+Q169E+Q172K+A174*+G182*+D183*+N195F+V206L+G255S+K391A+G476K, H1*+N54S+V56T+G109A+Q169E+Q172K+A174*+G182*+D183*+N195F+V206L+K269Q+K391A+G476K, H1*+N54S+V56T+G109A+A174S+G182*+D183*+N195F+V206L+G255S+K391A+G476K, H1*+N54S+V56T+G109A+Q169E+Q172K+A174S+G182*+D183*+N195F+V206L+K391A+G476K; wherein numbering is according to SEQ ID NO: 19.

In one embodiment, the invention relates to a polypeptide having alpha-amylase activity comprising an A and B domain obtained from the alpha-amylase comprising the amino acid sequence of SEQ ID NO: 3 which A and B domain is also disclosed herein as SEQ ID NO: 4. In one embodiment of the present invention, the amino acid sequence forming the A and B domain has at least 60% identity, such as at least 65%, such as at least 70%, such as at least 75% identity, such as at least 78%, at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 4.

In one embodiment, the invention relates to a polypeptide having alpha-amylase activity comprising a C domain donor is obtained from the alpha-amylase comprising the amino acid sequence of SEQ ID NO: 8 from which the C domain is determined to correspond to amino acids 398-483 which is also disclosed as SEQ ID NO: 8 herein. In one embodiment of the present invention, the amino acid sequence forming the C domain has at least 60% identity, such as at least 65%, such as at least 70%, such as at least 75% identity, such as at least 78%, at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 8.

In one embodiment of the present invention, the polypeptide having alpha-amylase activity comprising an A and B domain, and a C domain, wherein the amino acid sequence forming the A and B domain has at least 60% sequence identity to the amino acid sequence of SEQ ID NO: 4 and the amino acid sequence forming the C domain has at least 60% sequence identity to the amino acid sequence of SEQ ID NO: 8.

In one embodiment of the present invention, the amino acid sequence forming the A and B domain has at least 60% identity, such as at least 65%, such as at least 70%, such as 75% identity, such as at least 78%, at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 4, and the amino acid sequence forming the C domain has at least 60% identity to SEQ ID NO: 8.

In one embodiment of the present invention, the amino acid sequence forming the A and B domain has at least 75% identity, such as at least 78%, at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 4, and the amino acid sequence forming the C domain has at least 80% identity to SEQ ID NO: 8.

In one embodiment of the present invention, the amino acid sequence forming the A and B domain has at least 75% identity, such as at least 78%, at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 4, and the amino acid sequence forming the C domain has at least 85% identity to SEQ ID NO: 8.

In one embodiment of the present invention, the amino acid sequence forming the A and B domain has at least 75% identity, such as at least 78%, at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 4, and the amino acid sequence forming the C domain has at least 90% identity to SEQ ID NO: 8.

In one embodiment of the present invention, the amino acid sequence forming the A and B domain has at least 75% identity, such as at least 78%, at least 80%, at least 85%, at least 90%, at least 91%, at least 92%, at least 93%, at least 94%, at least 95%, at least 96%, at least 97%, at least 98%, at least 99% or 100% sequence identity to the amino acid sequence of SEQ ID NO: 4, and the amino acid sequence forming the C domain has at least 95% identity to SEQ ID NO: 8.

In another embodiment of the present invention, the amino acids corresponding to 181 and 182 in SEQ ID NO: 3 are deleted. In yet another embodiment of the invention, the amino acids corresponding to 183 and 184 in SEQ ID NO: 3 are deleted. In other embodiments the amino acids corresponding to 182 and 183 or 181 and 183 or 182 and 184 in SEQ ID NO: 3 are deleted. Thus, the invention also relates to a fusion polypeptide comprising the A and B domain of SEQ ID NO: 3 and the C domain from an alpha amylase of SEQ ID NO: 8 and further having a deletion of the amino acids corresponding to 183 and 184 in SEQ ID NO: 1. The invention further relates to a fusion polypeptide which is disclosed as SEQ ID NO: 9 herein.

In further preferred embodiments, the polypeptides of the present invention may further comprise an amino acid substitution in the C domain at one or more positions corresponding to positions 6, 22, 23 and 29 of the amino acid sequence of SEQ ID NO: 8.

In certain embodiments the polypeptide of the present invention comprises an amino acid substitution in the C domain at two positions corresponding to any of positions 6, 22, 23 and 29 of the amino acid sequence of SEQ ID NO: 8. In another embodiment the polypeptide of the present invention comprises an amino acid substitution at three positions corresponding to any of positions 6, 22, 23 and 29 of the amino acid sequence of SEQ ID NO: 8. In a preferred embodiment, the polypeptide of the present invention comprises an amino acid substitution at each position corresponding to positions 6, 22, 23 and 29 of the amino acid sequence of SEQ ID NO: 8. The amino acid substitution at position 6 may be any of 16A, C, D, E, F, G, H, K, L, M, N, P, Q, R, S, T, V, W or Y. Preferably it is 16L. The amino acid substitution at position 22 may be any of A22C, D, E, F, G, H, I, K, L, M, N, P, Q, R, S, T, V, W or Y. Preferably it is A22H. The amino acid substitution at position 23 may be any of A23C, D, E, F, G, H, I, K, L, M, N, P, Q, R, S, T, V, W or Y. Preferably it is A23P. The amino acid substitution at position 29 may be any of A29C, D, E, F, G, H, I, K, L, M, N, P, Q, R, S, T, V, W or Y. Preferably it is A29T.

In another embodiment of the present invention, the polypeptide comprises an A and B domain having at least 75% sequence identity to the A and B domain having the amino acid sequence of SEQ ID NO: 4, and further a C domain which has at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the C domain having the amino acid sequence of SEQ ID NO: 8. In a preferred embodiment the amino acid sequence making up the C domain comprises each of the substitutions corresponding to 16L, A22H, A23P and A29T of the amino acid sequence of SEQ ID NO: 8.

In a preferred embodiment the polypeptide of the invention comprises an A and B domain and a C domain wherein the amino acid sequence forming the A and B domain is at least 75% identical to the amino acid sequence of SEQ ID NO: 4 and comprises a deletion of amino acids corresponding to positions 183 and 184 of SEQ ID NO: 4 and the amino acid sequence forming the C domain is at least 75% identical to the amino acid sequence of SEQ ID NO: 8. In one embodiment the polypeptide comprises the substitutions corresponding to 16L, A22H, A23P and A29T of the amino acid sequence of SEQ ID NO: 8.

In another preferred embodiment the polypeptide of the invention comprises an A and B domain and a C domain wherein the amino acid sequence forming the A and B domain is at least 75% identical to the amino acid sequence of SEQ ID NO: 4 and comprises a deletion of amino acids corresponding to positions 181 and 182 of SEQ ID NO: 4 and the amino acid sequence forming the C domain is at least 75% identical to the amino acid sequence of SEQ ID NO: 8.

In another embodiment, the amino acid sequence of the A and B domain of the present invention comprises the sequence of SEQ ID NO: 4 and the amino acid sequence of the C domain comprises the sequence of SEQ ID NO: 8. In yet another embodiment the amino acid sequence of the A and B domain of the present invention consists of the sequence of SEQ ID NO: 4 and the amino acid sequence of the C domain consists of the sequence of SEQ ID NO: 8.

As also mentioned above, in a preferred embodiment of all the above-mentioned embodiments, the amino acids corresponding to 181+182 or 181+183 or 182+184 or 182+183 or 183+184 of SEQ ID NO: 4 are deleted. Preferable, amino acids 181+182 or 183+184 are deleted. It is most preferred that amino acids corresponding to 183+184 of SEQ ID NO: 4 are deleted.

Further, as also mentioned above, one or more of the amino acids corresponding to amino acids 6, 22, 23 and 29 of SEQ ID NO: 8 may be substituted. In one embodiment of the above-mentioned embodiments, the amino acid corresponding to the amino acid in position 6 of SEQ ID NO: 8 is substituted with a leucine. The amino acid corresponding to the amino acid in position 22 of SEQ ID NO: 8 may be substituted with a histidine. The amino acid corresponding to the amino acid in position 23 of SEQ ID NO: 8 may be substituted with a proline. The amino acid corresponding to the amino acid in position 29 of SEQ ID NO: 8 may be substituted with a threonine.

The alpha-amylases were originally generated and used due to their good performance at low temperature, but the present inventors have surprisingly discovered that these alpha-amylase variants also have good performance in low pH powder detergent compositions.

The invention is not limited to a particular method for determining alpha-amylase activity, but any recognized method for determining alpha-amylase activity may be used. One preferred method for determining alpha-amylase activity is based on cleavage of the PNP-G7 substrate. PNP-G7 is an abbreviation for 4,6-ethylidene(G₇)-p-nitrophenyl(G₁)-α,D-maltoheptaoside, a blocked oligosaccharide in the presence of an alpha-glucosidase. Following the cleavage, the alpha-glucosidase digest the hydrolysed substrate further to liberate a free PNP molecule which has a yellow color and thus can be measured by visible spectophometry at λ=405 nm (400-420 nm). Kits containing PNP-G7 substrate and alpha-glucosidase is manufactured by Roche/Hitachi (cat. No. 11876473).

In addition to the amino acid alterations specifically disclosed herein, an alpha-amylase variant in a composition of the invention may comprise additional alterations at one or more other positions. These additional alterations may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, in The Proteins, Academic Press, New York. Common conservative substitution groups include, but are not limited to: G=A=S; I=V=L=M; D=E; Y=F; and N=Q (where e.g. “G=A=S” means that these three amino acids may be substituted for each other).

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for alpha-amylase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

The present invention also relates to a powder detergent composition further comprising at least one or more detergent components.

In one embodiment, the composition further comprises at least one detergent component.

The choice of detergent component for a detergent composition is within the skill of the artisan and includes conventional ingredients, including the exemplary non-limiting components set forth below. The choice of components may include, for fabric care, the consideration of the type of fabric to be cleaned, the type and/or degree of soiling, the temperature at which cleaning is to take place, and the formulation of the detergent product.

In a particular embodiment, a detergent composition comprises a polypeptide having alpha-amylase and one or more non-naturally occurring detergent components, such as surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers. The detergent composition will typically comprise at least a surfactant and a builder.

In a particular embodiment, a detergent composition comprises a polypeptide having alpha-amylase and one or more naturally occurring detergent components, such as surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers. The detergent composition will typically comprise at least a surfactant and a builder.

In one embodiment, the composition further comprising one or more additional enzymes selected from the group consisting of proteases, lipases, cutinases, alpha-amylases, carbohydrases, cellulases, pectinases, mannanases, beta-amylase, pullulanase, perhydrolase, phospholipase arabinases, galactanases, xylanases, pectate lyase, galacturanase, hemicellulase, xyloglucanase, nucleases, lechinases, oxidases and mixtures thereof.

The detergent composition may e.g. be in the form of a regular or compact powder, a granulate, a homogeneous tablet, or a tablet having two or more layers.

The invention also relates to use of a composition of the present in a cleaning process, such as laundry or hard surface cleaning such as dish wash.

In one embodiment, the invention is directed to an ADW (Automatic Dish Wash) compositions comprising an alpha-amylase variant as disclosed in combination with one or more additional ADW composition components. The choice of additional components is within the skill of the artisan and includes conventional ingredients, including the exemplary non-limiting components set forth below.

In one embodiment the invention is directed to a laundry detergent composition comprising an alpha-amylase variant as disclosed in combination with one or more additional laundry detergent composition components. The choice of additional component is within the skills of the artisan and include conventional ingredients, including the exemplary non-limiting components set forth below.

In one embodiment, the polypeptide having alpha-amylase activity may be added to a detergent composition in an amount corresponding to 0.001-200 mg of enzyme protein per liter of wash liquor, preferably 0.005-50 mg of enzyme protein per liter of wash liquor, in particular 0.001-10 mg of enzyme protein per liter of wash liquor.

A granulated composition for laundry may for example include 0.001%-20%, such as 0.01%-10%, such as 0.05%-5% of enzyme protein by weight of the composition.
An automatic dish wash (ADW) composition may for example include 0.001%-30%, such as 0.01%-20%, such as 0.1-15%, such as 0.5-10% of enzyme protein by weight of the composition.
The detergent composition may be formulated into a granular detergent for laundry. Such detergent may e.g. comprise;
a) at least 0.001 mg alpha-amylase per gram of composition
b) anionic surfactant, preferably 5 wt % to 50 wt %
c) nonionic surfactant, preferably 1 wt % to 8 wt %
d) builder, preferably 5 wt % to 40 wt %, such as carbonates, zeolites, phosphate builder, calcium sequestering builders or complexing agents.
   Although components mentioned below are categorized by general header according to a particular functionality, this is not to be construed as a limitation, as a component may comprise additional functionalities as will be appreciated by the person skilled in the art.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized. Surfactants lower the surface tension in the detergent, which allows the stain being cleaned to be lifted and dispersed and then washed away.

When included therein, the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein, the detergent will usually contain from about 0% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein, the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein, the detergent will usually contain from about 0% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein, the detergent will usually contain from about 0% to about 10% by weight of a zwitterionic surfactant. Non-limiting examples of zwitterionic surfactants include betaine, alkyldimethylbetaine, sulfobetaine, and combinations thereof.

### Builders and Co-Builders

The detergent composition may contain about 0-65% by weight, such as about 5% to about 45% of a detergent builder or co-builder, or a mixture thereof. In a dish wash detergent, the level of builder is typically 40-65%, particularly 50-65%. Builders and chelators soften, e.g., the wash water by removing the metal ions form the liquid. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

In a preferred embodiment, the detergent composition is phosphate-free.

The detergent composition may also contain 0-20% by weight, such as about 5% to about 10%, of a detergent co-builder, or a mixture thereof. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra-(methylenephosphonic acid) (EDTMPA), diethylenetriaminepentakis (methylenephosphonic acid) (DTPMPA or DTMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N′, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 2009/102854 and U.S. Pat. No. 5,977,053.

The alpha-amylase variants of the invention may also be formulated into a dish wash composition, preferably an automatic dish wash composition (ADW), comprising:
a) at least 0.001 mg of active alpha-amylase according to the invention, and
b) 10-50 wt % builder preferably selected from citric acid, methylglycine-N,N-diacetic acid (MGDA) and/or glutamic acid-N,N-diacetic acid (GLDA) and mixtures thereof, and
c) at least one bleach component.

### Bleaching Systems

The detergent may contain 0-50% by weight, such as about 0.1% to about 25%, of a bleaching system. Bleach systems remove discolor often by oxidation, and many bleaches also have strong bactericidal properties, and are used for disinfecting and sterilizing. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator.

The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO 98/17767. A particular family of bleach activators of interest was disclosed in EP 624154 and particularly preferred in that family is acetyl triethyl citrate (ATC). ATC or a short chain triglyceride like triacetin has the advantage that it is environmentally friendly as it eventually degrades into citric acid and alcohol. Furthermore, acetyl triethyl citrate and triacetin have good hydrolytic stability in the product upon storage and are efficient bleach activators. Finally, ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst or a booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds, such as iron or cobalt complexes.

In some embodiments, the bleach component may be an organic catalyst selected from the group consisting of organic catalysts having the following formula:

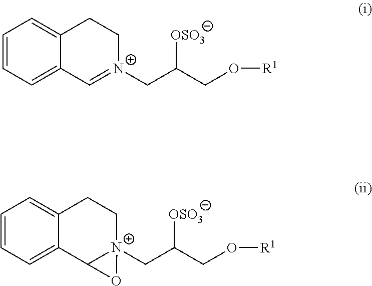

(iii) and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g., in WO 2007/087258, WO 2007/087244, WO 2007/087259 and WO 2007/087242. Suitable photobleaches may for example be sulfonated zinc phthalocyanine.

Preferably the bleach component comprises a source of peracid in addition to bleach catalyst, particularly organic bleach catalyst. The source of peracid may be selected from (a) pre-formed peracid; (b) percarbonate, perborate or persulfate salt (hydrogen peroxide souro) preferably in combination with a bleach activator; and (c) perhydrolase enzyme and an ester for forming peracid in situ in the presence of water in a textile or hard surface treatment step.

### Hydrotropes

A hydrotrope is a compound that solubilizes hydrophobic compounds in aqueous solutions (or oppositely, polar substances in a non-polar environment). Typically, hydrotropes have both hydrophilic and hydrophobic characters (so-called amphiphilic properties as known from surfactants); however, the molecular structures of hydrotropes generally do not favour spontaneous self-aggregation, see, e.g., review by Hodgdon and Kaler, 2007, Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behaviour, stability, and colloidal properties of systems containing substances of polar and non-polar character, including mixtures of water, oil, surfactants, and polymers. Hydrotropes are classically used across industries from pharma, personal care and food to technical applications. Use of hydrotropes in detergent compositions allows for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Fabric Hueing Agents

The detergent compositions of the present invention may also include fabric hueing agents such as dyes or pigments, which when formulated in detergent compositions can deposit onto a fabric when the fabric is contacted with a wash liquor comprising the detergent compositions and thus altering the tint of the fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/003274, WO 2005/003275, WO 2005/003276 and EP 1876226 (hereby incorporated by reference). The detergent composition preferably comprises from about 0.00003 wt. % to about 0.2 wt. %, from about 0.00008 wt. % to about 0.05 wt. %, or even from about 0.0001 wt. % to about 0.04 wt. % fabric hueing agent. The composition may comprise from 0.0001 wt % to 0.2 wt. % fabric hueing agent, this may be especially preferred when the composition is in the form of a unit dose pouch. Suitable hueing agents are also disclosed in, e.g., WO 2007/087257 and WO 2007/087243.

### Additional Enzymes

A detergent component or detergent composition may comprise one or more enzymes such as a proteases, lipases, cutinases, alpha-amylases, carbohydrases, cellulases, pectinases, mannanases, beta-amylase, pullulanase, perhydrolase, phospholipase arabinases, galactanases, xylanases, pectate lyase, galacturanase, hemicellulase, xyloglucanase, nucleases, lechinases, oxidases and mixtures thereof.

The properties of the selected enzyme(s) should be compatible with the selected detergent (e.g. pH-optimum, compatibility with other enzymatic and non-enzymatic ingredients, etc.).

### Cellulases

Suitable cellulases include mono-component and mixtures of enzymes of bacterial or fungal origin. Chemically modified or protein engineered mutants are also contemplated. The cellulase may for example be a mono-component or a mixture of mono-component endo-1,4-beta-glucanase also referred to as endoglucanase.

Suitable cellulases include those from the genera Bacillus, Pseudomonas, Humicola, Myceliophthora, Fusarium, Thielavia, Trichoderma, and Acremonium. Exemplary cellulases include a fungal cellulase from Humicola insolens (U.S. Pat. No. 4,435,307) or from Trichoderma, e.g. T. reesei or T. viride. Other suitable cellulases are from Thielavia e.g. Thielavia terrestris as described in WO 96/29397 or the fungal cellulases produced from Myceliophthora thermophila and Fusarium oxysporum disclosed in U.S. Pat. Nos. 5,648,263, 5,691,178, 5,776,757, WO 89/09259 and WO 91/17244. Also relevant are cellulases from Bacillus as described in WO 02/099091 and JP 2000210081. Suitable cellulases are alkaline or neutral cellulases having care benefits. Examples of cellulases are described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Carezyme®, Carezyme® Premium®, Celluzyme®, Celluclean®, Celluclast®, Endolase®, Renozyme®; Whitezyme® Celluclean® Classic, Cellusoft® (Novozymes A/S), Puradax®, Puradax HA, and Puradax EG (available from Genencor International Inc.) and KAC-500(B)™ (Kao Corporation).

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

A suitable peroxidase is preferably a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

Suitable peroxidases also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. The haloperoxidase may be a chloroperoxidase. Preferably, the haloperoxidase is a vanadium haloperoxidase, i.e., a vanadate-containing haloperoxidase. In a preferred method the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

The haloperoxidase may be derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

Suitable oxidases include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Proteases

Suitable proteases may be of any origin, but are preferably of bacterial or fungal origin, optionally in the form of protein engineered or chemically modified mutants. The protease may be an alkaline protease, such as a serine protease or a metalloprotease. A serine protease may for example be of the S1 family, such as trypsin, or the S8 family such as a subtilisin. A metalloprotease may for example be a thermolysin, e.g. from the M4 family, or another metalloprotease such as those from the M5, M7 or M35 families.

The term “subtilases” refers to a sub-group of serine proteases according to Siezen et al., Protein Eng. 4 (1991) 719-737 and Siezen et al., Protein Sci. 6 (1997) 501-523. Serine proteases are a subgroup of proteases characterized by having a serine in the active site, which forms a covalent adduct with the substrate. The subtilases may be divided into six subdivisions, the Subtilisin family, the Thermitase family, the Proteinase K family, the Lantibiotic peptidase family, the Kexin family and the Pyrolysin family.

Although proteases suitable for detergent use may be obtained from a variety of organisms, including fungi such as Aspergillus, detergent proteases have generally been obtained from bacteria and in particular from Bacillus. Examples of Bacillus species from which subtilases have been derived include Bacillus lentus, Bacillus alkalophilus, Bacillus subtilis, Bacillus amyloliquefaciens, Bacillus licheniformis, Bacillus pumilus and Bacillus gibsonii. Particular subtilisins include subtilisin lentus, subtilisin Novo, subtilisin Carlsberg, subtilisin BPN′, subtilisin 309, subtilisin 147 and subtilisin 168 and e.g. protease PD138 (described in WO 93/18140). Other useful proteases are e.g. those described in WO 01/16285 and WO 02/16547.

Examples of trypsin-like proteases include the Fusarium protease described in WO 94/25583 and WO 2005/040372, and the chymotrypsin proteases derived from Cellumonas described in WO 2005/052161 and WO 2005/052146.

Examples of metalloproteases include the neutral metalloproteases described in WO 2007/044993 such as those derived from Bacillus amyloliquefaciens, as well as e.g. the metalloproteases described in WO 2015/158723 and WO 2016/075078.

Examples of useful proteases are the protease variants described in WO 89/06279 WO 92/19729, WO 96/34946, WO 98/20115, WO 98/20116, WO 99/11768, WO 01/44452, WO 03/006602, WO 2004/003186, WO 2004/041979, WO 2007/006305, WO 2011/036263, WO 2014/207227, WO 2016/087617 and WO 2016/174234. Preferred protease variants may, for example, comprise one or more of the mutations selected from the group consisting of: S3T, V41, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V1021, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Q200L, Y203W, S206G, L211Q, L211D, N212D, N212S, M216S, A226V, K229L, Q230H, Q239R, N246K, S253D, N255W, N255D, N255E, L256E, L256D T268A and R269H, wherein position numbers correspond to positions of the Bacillus lentus protease shown in SEQ ID NO: 1 of WO 2016/001449. Protease variants having one or more of these mutations are preferably variants of the Bacillus lentus protease (Savinase®, also known as subtilisin 309) shown in SEQ ID NO: 1 of WO 2016/001449 or of the Bacillus amyloliquefaciens protease (BPN′) shown in SEQ ID NO: 2 of WO 2016/001449. Such protease variants preferably have at least 80% sequence identity to SEQ ID NO: 1 or to SEQ ID NO: 2 of WO 2016/001449.

Another protease of interest is the alkaline protease from Bacillus lentus DSM 5483, as described for example in WO 91/02792, and variants thereof which are described for example in WO 92/21760, WO 95/23221, EP 1921147, EP 1921148 and WO 2016/096711.

The protease may alternatively be a variant of the TY145 protease having SEQ ID NO: 1 of WO 2004/067737, for example a variant comprising a substitution at one or more positions corresponding to positions 27, 109, 111, 171, 173, 174, 175, 180, 182, 184, 198, 199 and 297 of SEQ ID NO: 1 of WO 2004/067737, wherein said protease variant has a sequence identity of at least 75% but less than 100% to SEQ ID NO: 1 of WO 2004/067737. TY145 variants of interest are described in e.g. WO 2015/014790, WO 2015/014803, WO 2015/014804, WO 2016/097350, WO 2016/097352, WO 2016/097357 and WO 2016/097354.

Examples of preferred proteases include:

(a) variants of SEQ ID NO: 1 of WO 2016/001449 comprising two or more substitutions selected from the group consisting of S9E, N43R, N76D, Q206L, Y209W, S259D and L262E, for example a variant with the substitutions S9E, N43R, N76D, V2051, Q206L, Y209W, S259D, N261W and L262E, or with the substitutions S9E, N43R, N76D, N185E, S188E, Q191N, A194P, Q206L, Y209W, S259D and L262E, wherein position numbers are based on the numbering of SEQ ID NO: 2 of WO 2016/001449;

(b) a variant of the polypeptide of SEQ ID NO: 1 of WO 2016/001449 with the mutation S99SE, wherein position numbers are based on the numbering of SEQ ID NO: 2 of WO 2016/001449;

(c) a variant of the polypeptide of SEQ ID NO: 1 of WO 2016/001449 with the mutation S99AD, wherein position numbers are based on the numbering of SEQ ID NO: 2 of WO 2016/001449;

(d) a variant of the polypeptide of SEQ ID NO: 1 of WO 2016/001449 with the substitutions Y167A+R170S+A194P, wherein position numbers are based on the numbering of SEQ ID NO: 2 of WO 2016/001449;

(e) a variant of the polypeptide of SEQ ID NO: 1 of WO 2016/001449 with the substitutions S9R+A15T+V68A+N218D+Q245R, wherein position numbers are based on the numbering of SEQ ID NO: 2 of WO 2016/001449;

(f) a variant of the polypeptide of SEQ ID NO: 1 of WO 2016/001449 with the substitutions S9R+A15T+G61E+V68A+A194P+V205+Q245R+N261 D, wherein position numbers are based on the numbering of SEQ ID NO: 2 of WO 2016/001449;

(g) a variant of the polypeptide of SEQ ID NO: 1 of WO 2016/001449 with the substitutions S99D+S101R/E+S103A+V1041+G160S; for example a variant of SEQ ID NO: 1 of WO 2016/001449 with the substitutions S3T+V41+S99D+S101E+S103A+V1041+G160S+V2051, wherein position numbers are based on the numbering of SEQ ID NO: 2 of WO 2016/001449;

(h) a variant of the polypeptide of SEQ ID NO: 2 of WO 2016/001449 with the substitutions S24G+S53G+S78N+S101N+G128A/S+Y217Q, wherein position numbers are based on the numbering of SEQ ID NO: 2 of WO 2016/001449;

(i) the polypeptide disclosed in GENESEQP under accession number BER84782, corresponding to SEQ ID NO: 302 in WO 2017/210295;

(j) a variant of the polypeptide of SEQ ID NO: 1 of WO 2016/001449 with the substitutions S99D+S101E+S103A+V1041+S156D+G160S+L262E, wherein position numbers are based on the numbering of SEQ ID NO: 2 of WO 2016/001449;

(k) a variant of the polypeptide of SEQ ID NO: 1 of WO 2016/001449 with the substitutions S9R+A15T+G61E+V68A+N76D+S99G+N218D+Q245R, wherein position numbers are based on the numbering of SEQ ID NO: 2 of WO 2016/001449;

(l) a variant of the polypeptide of SEQ ID NO: 1 of WO 2016/001449 with the substitutions V68A+S106A, wherein position numbers are based on the numbering of SEQ ID NO: 2 of WO 2016/001449; and

(m) a variant of the polypeptide of SEQ ID NO: 1 of WO 2004/067737 with the substitutions S27K+N109K+S111E+S171E+S173P+G174K+S175P+F180Y+G182A+L184F+Q198E+N199+T297P, wherein position numbers are based on the numbering of SEQ ID NO: 1 of WO 2004/067737.

A protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO2004/067737.

In one embodiment, the protease is a variant of the polypeptide of SEQ ID NO: 20 comprising the mutation S99AD, wherein position numbers correspond to positions of the polypeptide of SEQ ID NO: 21, for example a variant having at least 80%, at least 85%, at least 90%, at least 95%, at least 96%, at least 97% or at least 98% sequence identity to SEQ ID NO: 20. In one embodiment, the protease comprises or consists of the polypeptide of SEQ ID NO: 20 with the mutation S99AD.

In one embodiment, the protease is a variant of the polypeptide of SEQ ID NO: 20 comprising the mutation S99SE, wherein position numbers correspond to positions of the polypeptide of SEQ ID NO: 21, for example a variant having at least 80%, at least 85%, at least 90%, at least 95%, at least 96%, at least 97% or at least 98% sequence identity to SEQ ID NO: 21. In one embodiment, the protease comprises or consists of the polypeptide of SEQ ID NO: 20 with the mutation S99SE.

In one embodiment, the protease is a variant of the polypeptide of SEQ ID NO: 20 comprising the mutations Y167A+R170S+A194P, wherein position numbers correspond to positions of the polypeptide of SEQ ID NO: 21, for example a variant having at least 80%, at least 85%, at least 90%, at least 95%, at least 96%, at least 97% or at least 98% sequence identity to SEQ ID NO: 20. In one embodiment, the protease comprises or consists of the polypeptide of SEQ ID NO: 21 with the mutations Y167A+R170S+A194P.

In one embodiment, the protease is a variant of the polypeptide of SEQ ID NO: 20 comprising the mutations S9E+N43R+N76D+V205+Q206L+Y209W+S259D+N261W+L262E, wherein position numbers correspond to positions of the polypeptide of SEQ ID NO: 21, for example a variant having at least 80%, at least 85%, at least 90% or at least 95% sequence identity to SEQ ID NO: 20. In one embodiment, the protease comprises or consists of the polypeptide of SEQ ID NO: 20 with the mutations S9E+N43R+N76D+V205+Q206L+Y209W+S259D+N261W+L262E.

In one embodiment, the protease is a variant of the polypeptide of SEQ ID NO: 20 comprising the mutations S3T+V41+S99D+S101R+S103A+V1041+G160S+V199M+V205+L217D, wherein position numbers correspond to positions of the polypeptide of SEQ ID NO: 21, for example a variant having at least 80%, at least 85%, at least 90% or at least 95% sequence identity to SEQ ID NO: 20. In one embodiment, the protease comprises or consists of the polypeptide of SEQ ID NO: 20 with the mutations S3T+V41+S99D+S101R+S103A+V1041+G160S+V199M+V205+L217D.

In one embodiment, the protease is a variant of the polypeptide of SEQ ID NO: 20 comprising the mutations S3T+V41+S99D+S101E+S103A+V1041+G160S+V2051, wherein position numbers correspond to positions of the polypeptide of SEQ ID NO: 21, for example a variant having at least 80%, at least 85%, at least 90% or at least 95% sequence identity to SEQ ID NO: 20. In one embodiment, the protease comprises or consists of the polypeptide of SEQ ID NO: 20 with the mutations S3T+V41+S99D+S101E+S103A+V1041+G160S+V2051.

In one embodiment, the protease is a variant of the polypeptide of SEQ ID NO: 20 comprising the mutations S99D+S101E+S103A+V1041+G160S, wherein position numbers correspond to positions of the polypeptide of SEQ ID NO: 21, for example a variant having at least 80%, at least 85%, at least 90%, at least 95% or at least 96% sequence identity to SEQ ID NO: 20. In one embodiment, the protease comprises or consists of the polypeptide of SEQ ID NO: 20 with the mutations S99D+S101E+S103A+V1041+G160S.

In one embodiment, the protease is a variant of the polypeptide of SEQ ID NO: 20 comprising the mutations S99D+S101E+S103A+V1041+S156D+G160S+L262E, wherein position numbers correspond to positions of the polypeptide of SEQ ID NO: 21, for example a variant having at least 80%, at least 85%, at least 90% or at least 95% sequence identity to SEQ ID NO: 20. In one embodiment, the protease comprises or consists of the polypeptide of SEQ ID NO: 20 with the mutations S99D+S101E+S103A+V1041+S156D+G160S+L262E.

In one embodiment, the protease is a variant of the polypeptide of SEQ ID NO: 20 comprising the mutations S87N+S101G+V104N, wherein position numbers correspond to positions of the polypeptide of SEQ ID NO: 21, for example a variant having at least 80%, at least 85%, at least 90%, at least 95%, at least 96%, at least 97% or at least 98% sequence identity to SEQ ID NO: 20. In one embodiment, the protease comprises or consists of the polypeptide of SEQ ID NO: 20 with the mutations S87N+S101G+V104N.

In one embodiment, the protease comprises or consists of the polypeptide of SEQ ID NO: 21.

In one embodiment, the protease is a variant of the polypeptide of SEQ ID NO: 21 comprising the mutation Y217L, for example a variant having at least 80%, at least 85%, at least 90%, at least 95%, at least 96%, at least 97% or at least 98% sequence identity to SEQ ID NO: 21. In one embodiment, the protease comprises or consists of the polypeptide of SEQ ID NO: 21 with the mutation Y217L.

In one embodiment, the protease is a variant of the polypeptide of SEQ ID NO: 21 comprising the mutations S24G+S53G+S78N+S101N+G128S+Y217Q, for example a variant having at least 80%, at least 85%, at least 90%, at least 95% or at least 96% sequence identity to SEQ ID NO: 21. In one embodiment, the protease comprises or consists of the polypeptide of SEQ ID NO: 21 with the mutations S24G+S53G+S78N+S101N+G128S+Y217Q.

In one embodiment, the protease is a variant of the polypeptide of SEQ ID NO: 21 comprising the mutations S24G+S53G+S78N+S101N+G128A+Y217Q, for example a variant having at least 80%, at least 85%, at least 90%, at least 95% or at least 96% sequence identity to SEQ ID NO: 21. In one embodiment, the protease comprises or consists of the polypeptide of SEQ ID NO: 21 with the mutations S24G+S53G+S78N+S101N+G128A+Y217Q.

In one embodiment, the protease comprises or consists of the polypeptide of SEQ ID NO: 22.

In one embodiment, the protease is a variant of the polypeptide of SEQ ID NO: 22 having at least 80%, at least 85%, at least 90% or at least 95% sequence identity to SEQ ID NO: 22. The protease may e.g. be a variant of the polypeptide of SEQ ID NO: 22 comprising one or more mutations selected from the group consisting of S27K, N109K, S111E, S171E, S173P, G174K, S175P, F180Y, G182A, L184F, Q198E, N199K and T297P, for example 1, 2, 3, 4, 5, 6, 7, 8, 9, 10, 11, 12 or all of said mutations.

In one embodiment, the protease is a variant of the polypeptide of SEQ ID NO: 22 comprising the mutations S27K+N109K+S111E+S171E+S173P+G174K+S175P+F180Y+G182A+L184F+Q198E+N199K+T297P, for example a variant having at least 80%, at least 85%, at least 90% or at least 95% sequence identity to SEQ ID NO: 22. In one embodiment, the protease comprises or consists of the polypeptide of SEQ ID NO: 22 with the mutations S27K+N109K+S111E+S171E+S173P+G174K+S175P+F180Y+G182A+L184F+Q198E+N199K+T297P.

Suitable commercially available protease enzymes include those sold under the trade names Alcalase®, Duralase™, Durazym™, Relase®, Relase® Ultra, Savinase®, Savinase® Ultra, Primase™, Polarzyme®, Kannase®, Liquanase®, Liquanase® Ultra, Ovozyme®, Coronase®, Coronase® Ultra, Blaze®, Blaze Evity® 100T, Blaze Evity® 125T, Blaze Evity® 150T, Blaze Evity® 200T, Neutrase®, Everlase®, Esperase®, Progress® Uno, Progress® In and Progress® Excel (Novozymes A/S), those sold under the tradename Maxatase™, Maxacal™ Maxapem®, Purafect® Ox, Purafect® OxP, Puramax®, FN2™, FN3™, FN4^{ex}™, Excellase®, Excellenz™ P1000, Excellenz™ P1250, Eraser™, Preferenz® P100, Purafect Prime, Preferenz P110™, Effectenz P1000™, Purafect®, Effectenz P1050™, Purafect® Ox, Effectenz™ P2000, Purafast™, Properase®, Opticlean™ and Optimase® (Danisco/DuPont), BLAP (sequence shown in FIG. 29 of U.S. Pat. No. 5,352,604) and variants hereof (Henkel AG), and KAP (Bacillus alkalophilus subtilisin) from Kao.

### Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

### Amylases

The composition may comprise one or more additional alpha-amylases.

Suitable amylases which may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/19467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/10355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylases comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T491+G107A+H156Y+A181T+N190F+I201F+A209V+Q264S.

Other suitable amylases are amylases having the sequence of SEQ ID NO: 6 in WO 99/19467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/23873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/23873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 2008/153815, SEQ ID NO: 10 in WO 01/66712 or variants thereof having 90% sequence identity to SEQ ID NO: 2 of WO 2008/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 2009/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T1311, T1651, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165+K178L+T182G+Y305R+G475K,

wherein the variants are C-terminally truncated and optionally further comprise a substitution at position 243 and/or a deletion at position 180 and/or position 181.
Further suitable amylases are amylases having SEQ ID NO: 1 of WO 2013/184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, I203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or a deletion in position R178 and/or S179 or of T180 and/or G181. Most preferred amylase variants of SEQ ID NO: 1 comprise the substitutions: E187P+1203Y+G476K E187P+1203Y+R458N+T459S+D460T+G476K and optionally further comprise a substitution at position 241 and/or a deletion at position 178 and/or position 179.
Further suitable amylases are amylases having SEQ ID NO: 1 of WO 2010/104675 or variants having 90% sequence identity to SEQ ID NO: 1 thereof. Preferred variants of SEQ ID NO: 1 are those having a substitution, a deletion or an insertion in one of more of the following positions: N21, D97, V128K177, R179, S180, I181, G182, M200, L204, E242, G477 and G478.
More preferred variants of SEQ ID NO: 1 are those having the substitution in one of more of the following positions: N21D, D97N, V1281K177L, M200L, L204YF, E242QA, G477K and G478K and/or a deletion in position R179 and/or S180 or of 1181 and/or G182. Most preferred amylase variants of SEQ ID NO: 1 comprise the substitutions N21D+D97N+V1281, and optionally further comprise a substitution at position 200 and/or a deletion at position 180 and/or position 181.

Other suitable amylases are the alpha-amylase having SEQ ID NO: 12 in WO 01/66712 or a variant having at least 90% sequence identity to SEQ ID NO: 12. Preferred amylase variants are those having a substitution, a deletion or an insertion in one of more of the following positions of SEQ ID NO: 12 in WO 01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particularly preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078 and WO 2013/001087. Commercially available amylases include Duramyl™, Termamyl™, Fungamyl™, Stainzyme™, Stainzyme PIus™, Natalase™, Liquozyme X, BAN™ Amplify® and Amplify® Prime (from Novozymes A/S), and Rapidase™, Purastar™/Effectenz™ Powerase, Preferenz S1000, Preferenz S100, Preferenz S110 and Preferenz S210 (from Genencor International Inc./DuPont).

One preferred amylase is a variant of the amylase having SEQ ID NO: 13 in WO 2016/180748 with the alterations H1*+N54S+V56T+K72R+G109A+F113Q+R116Q+W167F+Q172G+A174S+G182*+D183*+G184T+N195F+V206L+K391A+P473R+G476K.

Another preferred amylase is a variant of the amylase having SEQ ID NO: 1 in WO 2013/001078 with the alterations D183*+G184*+W140Y+N195F+V206Y+Y243F+E260G+G304R+G476K.

Another preferred amylase is a variant of the amylase having SEQ ID NO: 1 in WO 2018/141707 with the alterations H1*+G7A+G109A+W140Y+G182*+D183*+N195F+V206Y+Y243F+E260G+N280S+G304R+E391A+G476K.

A further preferred amylase is a variant of the amylase having SEQ ID NO: 1 in WO 2017/191160 with the alterations L202M+T246V.

### Nucleases

Suitable nucleases include deoxyribonucleases (DNases) and ribonucleases (RNases) which are any enzyme that catalyzes the hydrolytic cleavage of phosphodiester linkages in the DNA or RNA backbone respectively, thus degrading DNA and RNA. There are two primary classifications based on the locus of activity. Exonucleases digest nucleic acids from the ends. Endonucleases act on regions in the middle of target molecules. The nuclease is preferably a DNase, which is preferable is obtainable from a microorganism, preferably a bacterium; in particular a DNase which is obtainable from a species of Bacillus is preferred; in particular a DNase which is obtainable from Bacillus cibi, Bacillus subtilis or Bacillus licheniformis is preferred. Examples of such DNases are described in WO 2011/098579, WO2014/087011 and WO2017/060475.

### Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants: The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant Science Series, volume 71, Marcel Dekker, Inc., 1997.

Dye Transfer Inhibiting Agents: The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent whitening agent: The detergent compositions of the present invention will preferably also contain additional components that may tint articles being cleaned, such as fluorescent whitening agent or optical brighteners. Where present the brightener is preferably at a level of about 0.01% to about 05%. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of: 4,4′-bis-(2-diethanolamino-4-anilino-s-triazin-6-ylamino) stilbene-2,2′-disulphonate; 4,4′-bis-(2,4-dianilino-s-triazin-6-ylamino) stilbene-2.2′-disulphonate; 4,4′-bis-(2-anilino-4(N-methyl-N-2-hydroxy-ethylamino)-s-triazin-6-ylamino) stilbene-2,2′-disulphonate, 4,4′-bis-(4-phenyl-2,1,3-triazol-2-yl)stilbene-2,2′-disulphonate; 4,4′-bis-(2-anilino-4(1-methyl-2-hydroxy-ethylamino)-s-triazin-6-ylamino) stilbene-2,2′-disulphonate and 2-(stilbyl-4″-naptho-1,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt. % to upper levels of 0.5 or even 0.75 wt. %.

Soil release polymers: The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 03/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents: The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent enzyme(s), i.e. alpha-amylase and optionally one or more additional enzymes, may be included in a detergent composition by adding separate additives containing one or more enzymes, or by adding a combined additive comprising these enzymes. A detergent additive comprising one or more enzymes can be formulated, for example, as a granulate, in particular a non-dusting granulate.

The detergent composition of the invention may be in any convenient form, e.g., a regular or compact powder, a granulate, a homogenous tablet, a tablet having two or more layers. The powder composition, e.g. powder, granulate or tablet, may also form part of a composite composition such as a compartment in a multiple compartment pouch or pod.

Pouches (pods) can be configured as single or multiple compartments and can be of any form, shape and material suitable to hold the composition, without allowing the release of the composition from the pouch prior to water contact. The pouch is made from water soluble film which encloses an inner volume. The inner volume can be divided into compartments of the pouch. Preferred films are polymeric materials, preferably polymers which are formed into a film or sheet. Preferred polymers, copolymers or derivates thereof are selected from polyacrylates, and water-soluble acrylate copolymers, methyl cellulose, carboxy methyl cellulose, sodium dextrin, ethyl cellulose, hydroxyethyl cellulose, hydroxypropyl methyl cellulose, maltodextrin, polymethacrylates, most preferably polyvinyl alcohol copolymers and hydroxypropyl methyl cellulose (HPMC). Preferably the level of polymer in the film for example PVA is at least about 60%. The preferred average molecular weight will typically be about 20,000 to about 150,000. Films can also be of blend compositions comprising hydrolytically degradable and water-soluble polymer blends such as polylactide and polyvinyl alcohol (known under the Trade reference M8630 as sold by Chris Craft In. Prod. of Gary, Ind., US) plus plasticizers like glycerol, ethylene glycerol, propylene glycol, sorbitol and mixtures thereof. The pouches can for example comprise a solid laundry detergent composition or part components and/or a liquid cleaning composition or part components separated by the water-soluble film. The compartment for liquid components can be different in composition than compartments containing solids. See, e.g., US 2009/0011970.

Detergent ingredients can be separated physically from each other by compartments in water dissolvable pouches or in different layers of tablet, thereby avoiding negative storage interaction between components. Different dissolution profiles of each of the compartments can also give rise to delayed dissolution of selected components in the wash solution.

### Granular Detergent Formulations

Enzymes in the form of granules, comprising an enzyme-containing core and optionally one or more coatings, are commonly used in granular (powder) detergents. Various methods for preparing the core are well-known in the art and include, for example, a) spray drying of a liquid enzyme-containing solution, b) production of layered products with an enzyme coated as a layer around a pre-formed inert core particle, e.g. using a fluid bed apparatus, c) absorbing an enzyme onto and/or into the surface of a pre-formed core, d) extrusion of an enzyme-containing paste, e) suspending an enzyme-containing powder in molten wax and atomization to result in prilled products, f) mixer granulation by adding an enzyme-containing liquid to a dry powder composition of granulation components, g) size reduction of enzyme-containing cores by milling or crushing of larger particles, pellets, etc., and h) fluid bed granulation. The enzyme-containing cores may be dried, e.g. using a fluid bed drier or other known method for drying granules in the feed or enzyme industry, to result in a water content of typically 0.1-10% w/w water.

The enzyme-containing cores are optionally provided with a coating to improve storage stability and/or to reduce dust formation. One type of coating that is often used for enzyme granulates for detergents is a salt coating, typically an inorganic salt coating, which may e.g. be applied as a solution of the salt using a fluid bed. Other coating materials that may be used are, for example, polyethylene glycol (PEG), methyl hydroxy-propyl cellulose (MHPC) and polyvinyl alcohol (PVA). The granules may contain more than one coating, for example a salt coating followed by an additional coating of a material such as PEG, MHPC or PVA.

For further information on enzyme granules and production thereof, see WO 2013/007594 as well as e.g. WO 2009/092699, EP 1705241, EP 1382668, WO 2007/001262, U.S. Pat. No. 6,472,364, WO 2004/074419 and WO 2009/102854.

### Microorganisms

The detergent components as well as the detergent composition may also comprise one or more microorganisms, such as one or more fungi, yeast, or bacteria.

In an embodiment, the one or more microorganisms are dehydrated (for example by lyophilization) bacteria or yeast, such as a strain of Lactobacillus.

In another embodiment, the microorganisms are one or more microbial spores (as opposed to vegetative cells), such as bacterial spores; or fungal spores, conidia, hypha.

Preferably, the one or more spores are Bacillus endospores; even more preferably the one or more spores are endospores of Bacillus subtilis, Bacillus licheniformis, Bacillus amyloliquefaciens, or Bacillus megaterium.

The microorganisms may be included in the detergent composition or components in the same way as enzymes.

The enzyme formulations, as well as the detergent formulations, may comprise one or more microorganisms or microbes. Generally, any microorganism(s) may be used in the enzyme/detergent formulations in any suitable amount(s)/concentration(s). Microorganisms may be used as the only biologically active ingredient, but they may also be used in conjunction with one or more of the enzymes described above.

The purpose of adding the microorganism(s) may, for example, be to reduce malodor as described in WO 2012/112718. Other purposes could include in-situ production of desirable biological compounds, or inoculation/population of a locus with the microorganism(s) to competitively prevent other non-desirable microorganisms form populating the same locus (competitive exclusion).

The term “microorganism” generally means small organisms that are visible through a microscope. Microorganisms often exist as single cells or as colonies of cells. Some microorganisms may be multicellular. Microorganisms include prokaryotic (e.g., bacteria and archaea) and eurkaryotic (e.g., some fungi, algae, protozoa) organisms. Examples of bacteria may be Gram-positive bacteria or Gram-negative bacteria. Example forms of bacteria include vegetative cells and endospores. Examples of fungi may be yeasts, molds and mushrooms. Example forms of fungi include hyphae and spores. Herein, viruses may be considered microorganisms.

Microorganisms may be recombinant or non-recombinant. In some examples, the microorganisms may produce various substances (e.g., enzymes) that are useful for inclusion in detergent compositions. Extracts from microorganisms or fractions from the extracts may be used in the detergents. Media in which microorganisms are cultivated or extracts or fractions from the media may also be used in detergents. In some examples, specific of the microorganisms, substances produced by the microorganisms, extracts, media, and fractions thereof, may be specifically excluded from the detergents. In some examples, the microorganisms, or substances produced by, or extracted from, the microorganisms, may activate, enhance, preserve, prolong, and the like, detergent activity or components contained with detergents.

Generally, microorganisms may be cultivated using methods known in the art. The microorganisms may then be processed or formulated in various ways. In some examples, the microorganisms may be desiccated (e.g., lyophilized). In some examples, the microorganisms may be encapsulated (e.g., spray drying). Many other treatments or formulations are possible. These treatments or preparations may facilitate retention of microorganism viability over time and/or in the presence of detergent components. In some examples, however, microorganisms in detergents may not be viable. The processed/formulated microorganisms may be added to detergents prior to, or at the time the detergents are used.

In one embodiment, the microorganism is a species of Bacillus, for example, at least one species of Bacillus selected from the group consisting of Bacillus subtilis, Bacillus amyloliquefaciens, Bacillus licheniformis, Bacillus atrophaeus, Bacillus pumilus, Bacillus megaterium, or a combination thereof. In a preferred embodiment, the aforementioned Bacillus species are on an endospore form, which significantly improves the storage stability.

### Uses

The present invention is also directed to methods for using the detergent compositions in laundering of textiles and fabrics, such as household laundry washing and industrial laundry washing.

The present invention further relates to the use of detergent composition according to the present invention in a cleaning process such as laundry, including industrial cleaning, ADW and hard surface cleaning. The soils and stains that are important for cleaning are composed of many different substances, and a range of different enzymes, all with different substrate specificities, have been developed for use in detergents both in relation to laundry and hard surface cleaning, such as dishwashing. These enzymes are considered to provide an enzyme detergency benefit, since they specifically improve stain removal in the cleaning process that they are used in, compared to the same process without enzymes. Stain removing enzymes that are known in the art include enzymes such as proteases, amylases, lipases, cutinases, cellulases, endoglucanases, xyloglucanases, pectinases, pectin lyases, xanthanases, peroxidaes, haloperoxygenases, catalases and mannanases.

In another aspect, the invention relates to a laundering process which may be for household laundering as well as industrial laundering. Furthermore, the invention relates to a process for the laundering of textiles (e.g. fabrics, garments, cloths etc.) where the process comprises treating the textile with a washing solution containing a detergent composition of the present invention. The laundering can for example be carried out using a household or an industrial washing machine or be carried out by hand using a detergent composition of the invention.

In another aspect, the invention relates to a dish wash process, including ADW; or hard surface cleaning, which may be for household cleaning as well as industrial cleaning. Furthermore, the invention relates to a process for dish wash or hard surface cleaning, where the process comprises treating the dishes or hard surfaces with a washing solution comprising a detergent composition of the present invention. The dish wash or hard surface cleaning can for example be carried out using a household dish washing machine or be carried out by hand using a detergent composition of the invention.

A detergent composition of the present invention may be formulated, for example, as a hand or machine laundry detergent composition including a laundry additive composition suitable for pre-treatment of stained fabrics and a rinse added fabric softener composition or be formulated as a detergent composition for use in general household hard surface cleaning operations or be formulated for hand or machine dishwashing operations.

The cleaning process or the textile care process may for example be a laundry process, a dishwashing process or cleaning of hard surfaces such as bathroom tiles, floors, table tops, drains, sinks and washbasins. Laundry processes can for example be household laundering but may also be industrial laundering. Furthermore, the invention relates to a process for laundering of fabrics and/or garments, where the process comprises treating fabrics with a washing solution containing a detergent composition of the invention. The cleaning process or a textile care process can for example be carried out in a machine washing or manually. The washing solution can for example be an aqueous washing solution containing a detergent composition.

In another aspect, the invention relates to a detergent composition comprising 5-100 g of a powder detergent comprising at least one polypeptide having alpha-amylase activity and further comprises one or more detergent components, as well as use thereof in a cleaning process, e.g. for laundry or dishwashing, wherein the composition has a pH of not more than about 9.0. In this aspect, the composition may e.g. comprise 8-80 g, such as 10-60 g of the powder detergent. In one embodiment, the detergent composition of this aspect is a compact composition, for example in the form of a highly compact powder or a tab, comprising e.g. 10-50 g, such as 10-40 g, such as 10-30 g or 10-20 g, of the powder detergent.

This aspect further relates to a method of cleaning, especially for cleaning fabrics or textiles, or for dishwashing, comprising contacting fabrics/textiles or dishes with the detergent composition of this aspect under conditions suitable for cleaning the fabrics/textiles or dishes.

The alpha-amylase in the composition according to this aspect, and for use thereof and a method of cleaning, may be any of the alpha-amylase described further above.

### Washing Method

The present invention provides a method of cleaning, especially for cleaning fabrics or textiles, or for dishwashing, with a detergent composition of the invention comprising an alpha-amylase.

The method of cleaning comprises contacting an object with a detergent composition comprising an alpha-amylase under conditions suitable for cleaning the object. In a preferred embodiment the detergent composition is used in a laundry or dish wash process.

Another embodiment relates to a method for removing stains from fabrics or textiles, which comprises contacting the fabric or textile with a composition of the invention under conditions suitable for cleaning the object.

Another embodiment relates to a method for removing stains from dishware, which comprises contacting the dishware with a composition of the invention under conditions suitable for cleaning the object.

The compositions may be employed at concentrations from about 100 ppm, preferably 500 ppm to about 15,000 ppm in solution. The water temperatures typically range from about 5° C. to about 95° C., including about 10° C., about 15° C., about 20° C., about 25° C., about 30° C., about 35° C., about 40° C., about 45° C., about 50° C., about 55° C., about 60° C., about 65° C., about 70° C., about 75° C., about 80° C., about 85° C. and about 90° C. The water to fabric ratio is typically from about 1:1 to about 30:1.

The enzyme(s) of the detergent composition of the invention may be stabilized using conventional stabilizing agents and protease inhibitors, e.g., a polyol such as propylene glycol or glycerol, a sugar or sugar alcohol, different salts such as NaCl; KCl; lactic acid, formic acid, boric acid, or a boric acid derivative, e.g., an aromatic borate ester, or a phenyl boronic acid derivative such as 4-formylphenyl boronic acid, or a peptide aldehyde such as di-, tri- or tetrapeptide aldehydes or aldehyde analogues (either of the form B1-B0-R wherein, R is H, CH3, CX3, CHX2, or CH2X (X=halogen), B0 is a single amino acid residue (preferably with an optionally substituted aliphatic or aromatic side chain); and B1 consists of one or more amino acid residues (preferably one, two or three), optionally comprising an N-terminal protection group, or as described in WO 2009/118375, WO 98/13459) or a protease inhibitor of the protein type such as RASI, BASI, WASI (bifunctional alpha-amylase/subtilisin inhibitors of rice, barley and wheat) or Cl2 or SSI. The composition may be formulated as described in, e.g., WO 92/19709, WO 92/19708 and U.S. Pat. No. 6,472,364. In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (Ill), Tin (II), cobalt (II), copper (II), Nickel (II), and oxovanadium (IV)).

The present invention is further described by the following examples that should not be construed as limiting the scope of the invention.

### EXAMPLES

### Materials and Methods

### PNP-G7 Assay

### Automatic Mechanical Stress Assay (AMSA) for Laundry

In order to assess the wash performance in laundry washing experiments are performed, using the Automatic Mechanical Stress Assay (AMSA). With the AMSA, the wash performance of a large quantity of small volume enzyme-detergent solutions can be examined. The AMSA plate has a number of slots for test solutions and a lid firmly squeezing the laundry sample, the textile to be washed against all the slot openings. During the washing time, the plate, test solutions, textile and lid are vigorously shaken to bring the test solution in contact with the textile and apply mechanical stress in a regular, periodic oscillating manner. For further description see WO02/42740 especially the paragraph “Special method embodiments” at page 23-24.
The laundry experiments are conducted under the experimental conditions specified below:

| | |
|---|---|
| Detergent dosage | 2.5 g/L (Laundry Powder Model Detergent 1) |
| | 5 g/L (Laundry Powder Model Detergent 2) |
| | 5.3 g/L (Laundry Powder Model Detergent 3) |
| | 5.25 g/L (Laundry Powder Model Detergent 4) |
| Enzyme dosage | 0.025-0.05-0.1-0.2 mg EP/L |
| Test solution volume | 160 microliters (140 microliters detergent and 20 |
| | microliters enzyme per slot) |
| pH | As is, measured to be: |
| | Laundry Powder Model Detergent 1: 8.3 |
| | Laundry Powder Model Detergent 2: 8.5 |
| | Laundry Powder Model Detergent 3: 8.6 |
| | Laundry Powder Model Detergent 4: 10.2 |
| Wash time | 20 minutes |
| Temperature | 40° C. and 20° C. |
| Water hardness | 15° dH |

Model detergents and test materials are as follows:

| | |
|---|---|
| Laundry Powder | Sodium citrate dihydrate 32.3% |
| Model Detergent 1 | Sodium-LAS 24.2% |
| | Sodium lauryl sulfate 32.2% |
| | Neodol 25-7 (alcohol ethoxylate) 6.4% |
| | Sodium sulfate 4.9% |
| Laundry Powder | Zeolite 43% |
| Model Detergent 2 | Sodium hydrogen carbonate 24% |
| | Sodium-LAS 18% |
| | Sodium lauryl sulfate 9.5% |
| | Neodol 25-7 (alcohol ethoxylate) 6% |
| Laundry Powder | Zeolite 19% |
| Model Detergent 3 | Sodium hydrogen carbonate 30% |
| | Sodium-LAS 15% |
| | Sodium sulfate 19% |
| | Sodium citrate 10% |
| | Alcohol ethylate 6% |
| | Soap 1% |
| Laundry Powder | LAS, sodium salt 11% |
| Model Detergent 4 | AS, sodium salt 1.8% |
| | Soap, sodium salt 2% |
| | AEO 3% |
| | Soda ash 15% |
| | Hydrous sodium silicate 3% |
| | Zeolite A 20% |
| | HEDP-Na4 0.13% |
| | Sodium citrate 2% |
| | PCA, copoly(acrylic acid/maleic acid), |
| | sodium salt 1.5% |
| | SRP 0.5% |
| | Sodium sulfate 39% |
| | Foam regulator 1% |
| Test Material | CS-28 (Rice starch on cotton) |

The following alpha-amylases were tested:

| Alpha- | |
|---|---|
| amylase | |
| number(s) | SEQ ID + mutations |
| Reference | SEQ ID NO: 2 + D183* + G184* + R118K + N195F + |
| | R320K + R458K |
| 1 | SEQ ID NO: 11 + D183* + G184* + W140Y + N195F + |
| | V206Y + Y243F + E260G + G304R + G476K |
| 2 | SEQ ID NO: 11 + H1* + G7A + G109A + W140Y + G182* |
| | + D183* + N195F + V206Y + Y243F + E260G + |
| | N280S + G304R + E391A + G476K |
| 3 | SEQ ID NO: 1 + L202M + T2456V |
| 4 | SEQ ID NO: 19 + H1* + N54S + V56T + K72R + |
| | G109A + F113Q + R116Q + W167F + Q172G + A174S + |
| | G182* + D183* + G184T + N195F + V206L + K391A + |
| | P473R + G476K |
| 5 | SEQ ID NO: 9 + H183* + G184* + I405L + A421H + |
| | A422P + A428T |

Test materials are obtained from Center For Testmaterials By, P.O. Box 120, 3133 KT Vlaardingen, the Netherlands.
Water hardness is adjusted to 15°dH by addition of CaCl₂, MgCl₂, and NaHCO₃ (Ca²⁺:Mg²⁺:NaHCO₃=4:1:7.5) to the test system. After washing the textiles were flushed in tap water and dried.
The wash performance is measured as the brightness of the colour of the textile washed. Brightness can also be expressed as the intensity of the light reflected from the sample when illuminated with white light. When the sample is stained the intensity of the reflected light is lower than that of a clean sample. Expressed another way, a cleaner sample will reflect more light and will have a higher intensity. Therefore, the intensity of the reflected light can be used to measure wash performance.
Color measurements are made with a professional flatbed scanner (Kodak iQsmart, Kodak, Midtager 29, DK-2605 Brondby, Denmark), which is used to capture an image of the washed textile.
To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r²+g²+b²}

### Example 1: Wash Performance of Different Amylase(s) in Different Model Detergents

The wash performance of alpha-amylase number 1 was investigated in different model detergents in AMSA as described above. The determined intensity values at 20° C. are shown in table 1 and determined intensity values at 40° C. are shown in table 2.

TABLE 1
Determined delta intensity values of Amylase variant relative to detergent
without amylase at 20° C. in different Laundry Model Detergents

| | 0.025 mgEP/L | | 0.05 mgEP/L | | 0.1 mgEP/L | | 0.2 mgEP/L | |
|---|---|---|---|---|---|---|---|---|
| Detergent | Amylase 4 | Amylase 5 | Amylase 4 | Amylase 5 | Amylase 4 | Amylase 5 | Amylase 4 | Amylase 5 |
| Laundry Powder | 20 | 8 | 22 | 11 | 26 | 18 | 26 | 20 |
| Model Detergent 1 | | | | | | | | |
| Laundry Powder | 15 | 5 | 18 | 9 | 21 | 17 | 26 | 23 |
| Model Detergent 2 | | | | | | | | |
| Laundry Powder | 14 | 12 | 18 | 10 | 20 | 15 | 23 | 17 |
| Model Detergent 3 | | | | | | | | |
| Laundry Powder | −3 | 5 | 2 | 11 | 3 | 14 | 2 | 14 |
| Model Detergent 4 | | | | | | | | |

TABLE 2
Determined delta intensity values of polypeptide having alpha-amylase relative
to detergent without amylase at 40° C. in different Laundry Model Detergents

| | 0.025 mgEP/L | | 0.05 mgEP/L | | 0.1 mgEP/L | | 0.2 mgEP/L | |
|---|---|---|---|---|---|---|---|---|
| Detergent | Amylase 4 | Amylase 5 | Amylase 4 | Amylase 5 | Amylase 4 | Amylase 5 | Amylase 4 | Amylase 5 |
| Laundry Powder | 25 | 20 | 32 | 28 | 36 | 32 | 36 | 36 |
| Model Detergent 1 | | | | | | | | |
| Laundry Powder | 13 | 20 | 25 | 24 | 29 | 31 | 36 | 35 |
| Model Detergent 2 | | | | | | | | |
| Laundry Powder | 20 | 23 | 27 | 27 | 29 | 33 | 34 | 37 |
| Model Detergent 3 | | | | | | | | |
| Laundry Powder | 1 | 0 | −2 | 5 | 2 | 9 | 1 | 19 |
| Model Detergent 4 | | | | | | | | |

From table 1 and 2 it is clear that the alpha-amylase number 1 shows significant wash performance in the low pH powder detergents Laundry Powder Model Detergent 1, 2 and 3, whereas no significant performance is detectable in the high pH powder detergent Laundry Powder Model Detergent 4, both at 20° C. and 4000.

### Example 2. Wash Performance Compared with Prior Art Alpha-Amylase

The wash performance of the polypeptide having alpha-amylase (average of two determination) was compared with the performance of reference polypeptide having alpha-amylase in low pH powder detergents Laundry Powder Model Detergent 1, 2 and 3 and in high pH powder detergent Laundry Powder Model Detergent 4, both at 20° C. and 40° C. The results were normalized so the results for the prior art alpha-amylase was set to 1.0
AMSA Results performed at 20° C. and 40° C. are shown in table 3 and table 4.

TABLE 3
Relative wash performance at 20° C.

| | Detergent | | | |
|---|---|---|---|---|
| | Laundry | Laundry | Laundry | Laundry |
| | Powder | Powder | Powder | Powder |
| | Model | Model | Model | Model |
| | Detergent 1 | Detergent 2 | detergent 3 | Detergent 4 |
| Reference | 1.00 | 1.00 | 1.00 | 1.00 |
| Alpha-amylase | | | | |
| Amylase 1 | 2.60 | 4.90 | 2.60 | 0.34 |
| Amylase 2 | 3.01 | 4.58 | 3.69 | 0.17 |
| Amylase 3 | 2.96 | 6.60 | 2.78 | 1.99 |
| Amylase 4 | 3.06 | 7.08 | 5.54 | 0.15 |
| Amylase 5 | 2.80 | 3.58 | 2.04 | 1.19 |

TABLE 4
Relative wash performance at 40° C.

| | Detergent | | | |
|---|---|---|---|---|
| | Laundry | Laundry | Laundry | Laundry |
| | Powder | Powder | Powder | Powder |
| | Model | Model | Model | Model |
| | Detergent 1 | Detergent 2 | detergent 3 | Detergent 4 |
| Reference | 1.00 | 1.00 | 1.00 | 1.00 |
| Alpha-amylase | | | | |
| Amylase 4 | 0.98 | 1.04 | 1.37 | 0.04 |
| Amylase 5 | 1.6 | 1.03 | 1.01 | 0.31 |

The data clearly show that the Amylase variant shows superior wash performance to the prior art alpha-amylase in low pH powder detergents, whereas a drastic performance drop is observed in high pH powder detergents, both at 20° C. and 40° C.

1. A powder detergent composition comprising at least one polypeptide having alpha-amylase activity and at least 60%, but less than 100% sequence identity to SEQ ID NOs: 1-19, wherein the composition has a pH value below 9.5, wherein pH is determined in a 5 g/l solution of the composition in deionized water at 20° C.

2. The composition according to claim 1, wherein the pH is in the range of 7.0-9.5; such as in the range of 7.5 to 9.0; such as in the range of 8.0 to 9.0.

3. The composition of claim 1, wherein the polypeptide having alpha-amylase activity has at least 60%, at least 65%, at least 70%, at least 75%, at least 80%, at least 85%, at least 90% or at least 95% but less than 100% sequence identity to SEQ ID NOs: 1-19.

4. The composition of claim 1, wherein the composition further comprises one or more detergent components.

5. The composition of claim 4, wherein detergent components selected from the group consisting of surfactants, hydrotropes, builders, co-builders, chelators or chelating agents, bleaching system or bleach components, polymers, fabric hueing agents, fabric conditioners, foam boosters, suds suppressors, dispersants, dye transfer inhibitors, fluorescent whitening agents, perfumes, optical brighteners, bactericides, fungicides, soil suspending agents, soil release polymers, anti-redeposition agents, enzyme inhibitors or stabilizers, enzyme activators, antioxidants, and solubilizers.

6. The composition of claim 5, wherein the builder is selected from a group consisting of phosphates, sodium citrate builders, sodium carbonate, sodium silicate, sodium and zeolites.

7. The composition of claim 6, wherein the surfactant is anionic and/or non-ionic.

8. The composition according to claim 7, wherein the anionic surfactant is selected from linear alkylbenzenesulfonates (LAS) isomers of LAS, alcohol ether sulfate (AEO, AEOS) and sodium lauryl ether sulfate and sodium laureth sulfate (SLES).

9. The composition according to claim 7, wherein the nonionic surfactant is selected from alcohol ethoxylates (AE or AEO), alcohol propoxylates, alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, N-acyl N-alkyl derivatives of glucosamine (glucamides, GA, or fatty acid glucamides, FAGA) and combinations thereof.

10. The composition of claim 1, which further comprises one or more additional enzymes selected from the group consisting of proteases, lipases, cutinases, alpha-amylases, carbohydrases, cellulases, pectinases, mannanases, beta-amylase, pullulanase, perhydrolase, phospholipase arabinases, galactanases, xylanases, pectate lyase, galacturanase, hemicellulase, xyloglucanase, nucleases, lechinases, oxidases and mixtures thereof.

11. The composition of claim 1, which is a laundry or dish wash composition.

12. The composition according to claim 11, wherein the dishwash composition is as an Automatic Dish Wash (ADW) detergent composition, a soap bar, or powder dish wash composition, such as an ADW unit dose detergent composition and such as a Hand Dish Wash (HDW) detergent composition.

13. The composition of any of claim 11, wherein the composition is a unit dosage form such as a tab or pouch.

14. (canceled)

15. A method of cleaning, especially for cleaning fabrics or textiles, or for dishwashing, comprising contacting fabrics/textiles or dishes with a detergent composition of claim 1 under conditions suitable for cleaning the fabrics/textiles or dishes.

16. A method of cleaning laundry or a hard surface comprising contacting the laundry or hard surface with a detergent composition of claim 1 under conditions suitable for cleaning the laundry or hard surface.
